(19) United States
(12) Patent Application Publication
(10) Pub. No.: US 2007/0244381 A1
(43) Pub. Date: Oct. 18, 2007

(54) Blood Analyte Determinations

(76) Inventors: Mark Ries Robinson, Albuquerque, NM (US); Mike Borrello, Carlsbad, CA (US); Richard Thompson, Dana Point, CA (US); Stephen Vanslyke, Carlsbad, CA (US); Steve Bernard, Andover, MN (US); John O'Mahony, Maple Grove, MN (US)

Correspondence Address: V. Gerald Grafe, esq., P.O. Box 2689, Corrales, NM, 87048, US

(21) Appl. No.: 11/679,826

(22) Filed: Feb. 27, 2007

Related U.S. Application Data
(60) Provisional application No. 60/791,719, filed on Apr. 12, 2006.

(51) Int. Cl. A61B 5/157

(52) U.S. Cl. 600/365

(57) ABSTRACT

The present invention comprises methods and apparatuses that can provide measurement of glucose and other analytes with a variety of sensors without many of the performance-degrading problems of conventional approaches. An apparatus according to the present invention comprises a blood access system, adapted to remove blood from a body and infuse at least a portion of the removed blood back into the body. Such an apparatus also comprises an analyte sensor, mounted with the blood access system such that the analyte sensor measures the analyte in the blood that has been removed from the body by the blood access system. A method according to the present invention comprises removing blood from a body, using an analyte sensor to measure an analyte in the removed blood, and infusing at least a portion of the removed blood back into the body. The use of a non-contact sensor with a closed system creates a system will minimal infection risk.

### CROSS-REFERENCE TO RELATED APPLICATIONS

This application claims priority to U.S. provisional application 60/791,719, filed Apr. 12, 2006, incorporated herein by reference, and as a continuation in part of international application PCT/US2006/060850 designating the U.S., which international application claimed priority to U.S. provisional application 60,737,254, filed Nov. 15, 2005, incorporated herein by reference.

### FIELD OF THE INVENTION

This invention relates to the field of the measurement of blood analytes and more specifically to the measurement of analytes such as glucose in blood that has been temporarily removed from a body.

### BACKGROUND OF THE INVENTION

More than 20 peer-reviewed publications have demonstrated that tight control of blood glucose significantly improves critical care patient outcomes. Tight glycemic control (TGC) has been shown to reduce surgical site infections by 60% in cardiothoracic surgery patients and reduce overall ICU mortality by 40% with significant reductions in ICU morbidity and length of stay. See, e.g., Furnary Tony. Oral presentation at 2005 ADA annual, session titled “Management of the Hospitalized Hyperglycemic Patient;” Van den Berghe et at, NEJM 2001; 345:1359. Historically, caregivers have treated hyperglycemia (high blood glucose) only when glucose levels exceeded 220 mg/dl. Based upon recent clinical findings, however, experts now recommend IV insulin administration to control blood glucose to within the normoglycemic range (80-110 mg/dl). Adherence to such strict glucose control regimens requires near-continuous monitoring of blood glucose and frequent adjustment of insulin infusion to achieve normoglycemia while avoiding risk of hypoglycemia (low blood glucose). In response to the demonstrated clinical benefit, approximately 50% of US hospitals have adopted some form of tight glycemic control with an additional 23% expected to adopt protocols within the next 12 months. Furthermore, 36% of hospitals already using glycemic management protocols in their ICUs plan to expand the practice to other units and 40% of hospitals that have near-term plans to adopt TGC protocols in the ICU also plan to do so in other areas of the hospital.

Given the compelling evidence for improved clinical outcomes associated with tight glycemic control, hospitals are under pressure to implement TGC as the standard of practice for critical care and cardiac surgery patients. Clinicians and caregivers have developed TGC protocols that use IV insulin administration to maintain normal patient glucose levels. To be safe and effective these protocols require frequent blood glucose monitoring. Currently, these protocols involve periodic removal of blood samples by nursing staff and testing on handheld meters or blood gas analyzers. Although hospitals are responding to the identified clinical need, adoption has been difficult with current technology due to two principal reasons,

Fear of hypolycemia. The target glucose range of 80-110 mg/dl brings the patient near clinical hypoglycemia (blood glucose less than 50 mg/dl). Patients exposed to hypoglycemia for greater than 30 minutes have significant risk of neurological damage. IV insulin administration with only intermittent glucose monitoring (typically hourly by most TGC protocols) exposes patients to increased risk of hypoglycemia. In a recent letter to the editors of Intensive Care medicine, it was noted that 42% of patients treated with a TOGC protocol in the UK experienced at least one episode of hypoglycemia. See, e.g., Iain Mackenzie et at., “Tight glycemic control: a survey of intensive care practice in large English Hospitals,” Intensive Care Med (2005) 31:1136. In addition, handheld meters require procedural steps that are often cited as a source of measurement error, further exacerbating the fear (and risk) of accidentally taking the blood glucose level too low. See, e.g., Bedside Glucose Testing systems, CAP today, April 2005, page 44.

Burdensome procedure. Most glycemic control protocols require frequent glucose monitoring and insulin adjustment at 30 minute to 2 hour intervals (typically hourly) to achieve normoglycemia. Caregivers recognize that glucose control would be improved with continuous or near-continuous monitoring. Unfortunately, existing glucose monitoring technology is incompatible with the need to obtain frequent measurements. Using current technology, each measurement requires removal of a blood sample, performance of the blood glucose test, evaluation of the result, determination of the correct therapeutic action, and finally adjustment to the insulin infusion rate. High measurement frequency requirements coupled with a labor-intensive and time-consuming test places significant strain on limited ICU nursing resources that already struggle to meet patient care needs.

Development of Continuous Glucose Monitors. There has been significant effort devoted to the development of in-vivo glucose sensors that continuously and automatically monitor an individual's glucose level. Such a device would enable individuals to more easily monitor their glucose light levels. Most of the efforts associated with continuous glucose monitoring have been focused on subcutaneous glucose measurements. In these systems, the measurement device is implanted in the tissue of the individual. The device then reads out a glucose concentration based upon the glucose concentration of the fluid in contact with the measurement device. Most of the systems implant the needle in the subcutaneous space and the fluid measured under measurement is interstitial fluid.

As used herein, a “contact glucose sensor is any measurement device that makes physical contact with the fluid containing the glucose under measurement. Standard glucose meter,s are an example of a contact glucose sensor. In use a drop of blood is placed on a disposable strip for the determination of glucose. An example of a glucose sensor is an electrochemical sensor. An electrochemical sensor is a device configured to detect the presence and/or measure the level of analyte in a sample via electrochemical oxidation and reduction reactions on the sensor. These reactions are transduced to a electrical signal that can be correlated to an amount, concentration, or level of analyte in the sample. Another example of a glucose sensor is a microfluidic chip or micro post technology. These chips are a small device with micro-sized posts arranged in varying numbers on a rectangle array of specialized material which can measure chemical concentrations. The tips of the microposts can be coated with a biologically active layer capable of measuring concentrations of specific lipids, proteins, antibodies, toxins and sugars. Microposts have been made of Foturan, a photo defined glass. Another example of a glucose sensor is a fluorescent measurement technology. The system for measurement is composed of a fluorescence sensing device consisting of a light source, a detector, a fluorophore (fluorescence dye), a quencher and an optical polymer matrix. When excited by light of appropriate wavelength, the fluorophore emits light (fluoresces). The intensity of the light or extent of quenching is dependent on the concentration of the compounds in the media. Another example of a glucose sensor is an enzyme based monitoring system that includes a sensor assembly, and an outer membrane surrounding the sensor. Generally, enzyme based glucose monitoring systems use glucose oxidase to convert glucose and oxygen to a measurable end product. The amount of end product produced is proportional to the glucose concentration. Ion specific of electrodes are another example of a contact glucose sensor.

As used herein, a “glucose sensor” is a noncontact glucose sensor, a contact glucose sensor, or any other instrument or technique that can determine the glucose presence or concentration of a sample. As used herein, a “noncontact glucose sensor” is any measurement method that does not require physical contact with the fluid containing the glucose under measurement. Example noncontact glucose sensors include sensors based upon spectroscopy. Spectroscopy is a study of the composition or properties of matter by investigating light, sound, or particles that are emitted, absorbed or scattered by the matter under investigation. Spectroscopy can also be defined as the study of the interaction between light and matter. There are three main types of spectroscopy: absorption spectroscopy, emission spectroscopy, and scattering spectroscopy. Absorbance spectroscopy uses the range of the electromagnetic spectrum in which a substance absorbs. After calibration, the amount of absorption can be related to the concentration of various compounds through the Beer-Lambert law. Emission spectroscopy uses the range of the electromagnetic spectrum in which a substance radiates. The substance first absorbs energy and then I radiates this energy as light This energy can be from a variety of sources including collision and chemical reactions. Scattering spectroscopy measure certain physical characteristics or properties by measuring the amount of light that a substance scatters at certain wavelengths, incidence angles and polarization angles. One of the most useful applications of light scattering spectroscopy is Raman spectroscopy but polarization spectroscopy has also been used for analyte measurements. There are many types of spectroscopy and the list below describes several types but should not be considered a definitive list. Atomic Absorption Spectroscopy is where energy absorbed by the sample is used to assess its characteristics. Sometimes absorbed energy causes light to be released from the sample, which may be measured by a technique such as fluorescence spectroscopy. Attenuated Total Reflectance Spectroscopy is used to sample liquids where the sample is penetrated by an energy beam one or more times and the reflected energy is analyzed. Attenuated total reflectance spectroscopy and the related technique called frustrated multiple internal reflection spectroscopy are used to analyze liquids. Electron Paramagnetic Spectroscopy is a microwave technique based on splitting electronic energy fields in a magnetic field. It is used to determine structures of samples containing unpaired electrons. Electron Spectroscopy includes several types of electron spectroscopy, all associated with measuring changes in electronic energy levels. Gamma-ray Spectroscopy uses Gamma radiation as the energy source in this type of spectroscopy, which includes activation analysis and Mossbauer spectroscopy. Infrared Spectroscopy uses the infrared absorption spectrum of a substance, sometimes called its molecular fingerprint. Although frequently used to identify materials, infrared spectroscopy also is used to quantify the number of absorbing molecules. Types of spectroscopy include the use of mid-infrared light, near-infrared light and uv/visible light. Fluorescence spectroscopy uses photons to excite a sample which will then emit lower energy photons. This type of spectroscopy has become popular in biochemical and medical applications. It can be used with confocal microscopy, fluorescence resonance energy transfer, and fluorescence lifetime imaging. Laser Spectroscopy can be used with many spectroscopic techniques to include absorption spectroscopy, fluorescence spectroscopy, Raman spectroscopy, and surface-enhanced Raman spectroscopy. Laser spectroscopy provides information about the interaction of coherent light with matter. Laser spectroscopy generally has high resolution and sensitivity. Mass Spectrometry uses a mass spectrometer source to produce ions. Information about a sample can be obtained by analyzing the dispersion of ions when they interact with the sample, generally using the mass-to-charge ratio. Multiplex or Frequency-Modulated Spectroscopy is a type of spectroscopy where each optical wavelength that is recorded is encoded with a frequency containing the original wavelength information. A wavelength analyzer can then reconstruct the original spectrum. Hadamard spectroscopy is another type of multiplex spectroscopy. Raman spectroscopy uses Raman scattering of light by molecules to provide information on a sample's chemical composition and molecular structure. X-ray Spectroscopy is a technique involving excitation of inner electrons of atoms, which may be seen as x-ray absorption. An x-ray fluorescence emission spectrum can be produced when an electron falls from a higher energy state into the vacancy created by the absorbed energy. Nuclear magnetic resonance spectroscopy analyzes certain atomic nuclei to determine different local environments of hydrogen, carbon and other atoms in a molecule of an organic compound. Grating or dispersive spectroscopy typically records individual groups of wavelengths. As can be seen by the number of methods, there are multiple methods and means for measuring glucose in a non-contact mode.

Note that the glucose sensors are referred to via a variety of nomenclature and terms throughout the medical literature. As examples, glucose sensors are referred to in the literature as ISF microdialysis sampling and online measurements, continuous alternate site measurements, ISF fluid measurements, tissue glucose measurements, 1SF tissue glucose measurements, body fluid measurements, skin measurement, skin glucose measurements, subcutaneous glucose measurements, extracorporeal glucose sensors, in-vivo glucose sensors, and ex-vivo glucose sensors. Examples of such systems include those described in U.S. Pat. No. 6,990,366 Analyte Monitoring Device and Method of Use; U.S. Pat. No. 6,259,937 Implantable Substrate Sensor; U.S. Pat. No. 6,201,980 Implantable Medical Sensor System; U.S. Pat. No. 6,477,395 Implantable in Design Based Monitoring System Having Improved Longevity Due to in Proved Exterior Surfaces; U.S. Pat. No. 6,653,141 Polyhydroxyl-Substituted organic Molecule Sensing Method and Device; US patent application 20050095602 Microfluidic Integrated Microarrays For Biological Detection; each of the preceding incorporated by reference herein.

In the typical use of the above glucose sensors require calibration before and during use. The calibration process generally involves taking a conventional technology (e.g., fingerstick) measurement and correlating this measurement with the sensors current output or measurement. This type of calibration procedure helps to remove biases and other artifacts associated with the implantation of the sensor in the body. The process is done upon initiation of use and then again during the use of the device.

Testing of CGMS systems in the ICU setting. Since continuous glucose monitoring systems (CGMS) provide a continuous glucose measurement, it can be desirable to use these types of systems for implementation of tight glycemic control protocols. The use of a continuous glucose monitoring systems has been investigated by several clinicians. These investigations have generally taken two different forms. The first has been to use the continuous glucose monitors in the standard manner of placing them in the tissue such that they measure interstitial glucose. A second avenue of investigation has used the sensors in direct contact with blood via an extracorporeal blood loop. Summary information from existing publications is presented below.

“Experience with continuous glucose monitoring system a medical intensive care unit”, by Goldberg at al, Diabetes Technology and Therapeutics, Volume 6, Number 3, 2004. FIG. 1 shows the scatter plot of the 542 paired glucose measurements. For these measurements the r value was 0.88 overall with 63.4% of the measurement pairs fell within 20 mg/dl of one another while 87.8% fell within 40 mg/dl. Additionally the authors state that seven of the 41 sensors (17%) exhibited persistent malfunction prior to the study end point of 72 hours.

“The use of two continuous glucose sensors during and after surgery” by Vriesendorp et al., Diabetes Technology and Therapeutics, Volume 7, Number 2, 2005. In a summary conclusion the authors' state that the technical performance and accuracy of continuous glucose sensors need improvement before continuous glucose can sensors can be used to implement strict glycemic control protocols during and after surgery.

“Closed loop glucose control in critically ill patients using continuous glucose monitoring system in real-time”, by Chee et al, IEEE transactions on information technology in biomass and, volume 7, Number one, March 2003. The authors provide a summary comment that improvement of real-time sensor accuracy is needed. In fact the actual accuracy of the results generated showed that 64.6% of the sensor readings would be clinically accurate (zone b) while 2.88% would lead to in no treatment (zone b), as illustrated in FIG. 2. The authors state that the accuracy of subcutaneously measured glucose is dependent “on equilibration of glucose concentration to be reached before ISF, plasma and whole blood, taking into account a possible time delay. Skin perfusion on the site of the sensor insertion differs from patient to patient. Most patients admitted to the ICU have a degree of peripheral edema and glucose monitoring based on ISF readings under such conditions would be subjected to variation in ISF-plasma—whole blood equilibration. The problem is likely exacerbated by non-ambulatory patients with little dynamic circulation of ISF in the subcutaneous space.

Problems with Existing CGMS. The present invention can address various problems recognized in the use of CGMS. The performance of existing CGMS when placed in the tissue or an extracorporeal blood circuit is limited. The source of the performance limitation can be segmented into several discrete error sources. The first is associated with the actual performance of the sensor overtime, while the second error grouping is associated with the physiology assumptions needed for accurate measurements.

General performance limitations: in a simplistic sense electrochemical or enzyme based sensors use glucose oxidase to convert glucose and oxygen to gluconic acid and hydrogen peroxide. An electrochemical oxygen detector is then employed to measure the concentration of remaining oxygen after reaction of the glucose; thereby providing an inverse measure of the glucose concentration. A second enzyme, or catalyst, is optimally included with the glucose oxidase to catalyze the decomposition of the hydrogen peroxide to water, in order to prevent interference in measurements from the hydrogen peroxide. In operation the system of measuring glucose requires that glucose be the rate limiting reagent of the enzymatic reaction. When the glucose measurement system is used in conditions where the concentration of oxygen can be limited a condition of “oxygen deficiency” can occur in the area of the enzymatic portion of the system and results in an inaccurate determination of glucose concentration. Further, such an oxygen deficit contributed other performance related problems for the sensor assembly, including diminished sensor responsiveness and undesirable electrode sensitivity. Intermittent inaccuracies can occur when the amount of oxygen present at the enzymatic sensor varies and creates conditions where the amount of oxygen can be rate limiting. This is particularly problematic when seeking the use the sensor technology on patients with cardiopulmonary compromise. These patients are poorly perfused and may not have adequate oxygenation.

Performance over time: in many conditions an electrochemical sensor shows drift and reduced sensitivity over time. This alteration in performance is due to a multitude of issues which can include: coating of the sensor membrane by albumin and fibrin, reduction in enzyme efficiency, oxidation of the sensor and a variety of other issues that are not completely understood. As a result of these alterations in sensor performance the sensors must be recalibrated on a frequent basis. The calibration procedure typically requires the procurement of a blood measurement and a correlation of this measurement with the sensor performance, if a bias or difference is present the implanted sensor's output is modified so that there is agreement between the value reported by the sensor and the blood reference. This process requires a separate, external measurement technique and is quite cumbersome to implement.

Physiological assumptions, for the sensor to effectively represent blood glucose values a strong correlation between the glucose levels in blood and subcutaneous interstitial fluid must exist. If this relationship does not exist, a systematic error will be inherent in the sensor signal with potentially serious consequences. A number of publications have shown a close correlation between glucose levels in blood and subcutaneous interstitial fluid. However, most of these investigations were performed under steady-state conditions only, meaning slow changes in blood glucose (<1 mg/dl/min). This restriction on the rate of change is very relevant due to the compartmentalization that exists between the blood and interstitial fluid. Although there is free exchange of glucose between plasma and interstitial fluid, a change in blood glucose will not be immediately accompanied by an immediate change of the interstitial fluid glucose under dynamic conditions. There is a so-called physiological lag time. The physiological lag time is influenced by many parameters, including the overall perfusion of the tissue. In conditions where tissue perfusion is poor and the rate of glucose change is significant the physiological lag can become very significant. In these conditions the resulting difference between interstitial glucose and blood glucose can become quite large. As noted above the overall cardiovascular or perfusion status of the patient can have significant influence on the relationship between ISF glucose and whole blood glucose. Since patients in the intensive care unit or operating room typically have some type of cardiovascular compromise the needed agreement between ISF glucose and whole blood is not present.

Additional understanding with respect to the calibration of continuous glucose monitors can be obtained from the following references. U.S. Pat. No. 7,029,444, Real-Time Self Adjusting Calibration Algorithm. The patent defines a method of calibrating glucose monitor data that utilizes to reference glucose values from a reference source that has a temporal relationship with the glucose monitor data. The method enables calibrating the calibration characteristics using the reference glucose values and the corresponding glucose monitor data. US patent application 200510143636 System and Method for Sensor Recalibration. The patent application described a methodology for sensor recalibration utilizing an array of data which includes historical as well as recent data, such as, blood glucose readings and sensor electrode readings. The state in the application, the accuracy of the sensing system is generally limited by the drift characteristics of the sensing element over time and the amount of environmental noise introduced into the output of the sensing element. To accommodate the inherent drift in the sensing element in the noise inherent in the system environment the sensing system is periodically calibrated or recalibrated.

Additional understanding with respect to sensor drift can be obtained from the following references. Article by Gough et al. in Two-Dimensional Enzyme Electrode Sensor for Glucose, Vol. 57, Analytical Chemistry pp 2351 et seq (1985). U.S. Pat. No. 6,477,395 Implantable Enzyme-based Monitoring System Having Improved Longevity Due to Improved Exterior Surfaces. The patent describes an implantable enzyme based monitoring system having an outer membrane that resists blood coagulation and protein binding. In the background of the invention, columns 1 and 2 the authors describe in detail the limitations and problems associated with enzyme-based glucose monitoring systems.

The operation of many of the embodiments disclosed herein involves the use of a maintenance fluid. A maintenance fluid is a fluid used in the system for any purpose. Fluids can include saline, lactated ringers, mannitol, amicar, isolyte, heta starch, blood, plasma, serum, platelets, or any other fluid that is infused into the patient. In addition to fluids that are infused into the patient, maintenance fluids can include fluids specifically used for calibrating the device or for cleaning the system, for other diagnostic purposes, and/or can include fluids that perform a combination of such functions.

Glucose sensors, both contact and noncontact, have different capabilities with respect to making accurate measurements in moving blood. For example, most strip based measurement technologies require an enzymatic reaction with blood and therefore have an operation incompatible with flowing blood. Other sensors can operate in a mode of establishing a constant output in the presence of flowing blood. Noncontact optical or spectroscopic sensors are especially applicable to conditions where the blood is flowing by the fact that they do not require an enzymatic reaction. For the blood access system described herein, one objective is to develop a system that does not result in blood clotting. Generally speaking blood that is stagnant is more prone to clotting than blood that is moving. Therefore the use of measurement systems that do not require stationery blood is beneficial. This benefit is especially relevant if the blood is to be re-infused into the patient.

In an instrument that operates in the intensive care unit on critically ill patients infection risk is an important consideration. A closed system is typically desired as the system has no mechanism for external entry into the flow path after initial set-up and during operation. The system can function without any opening or closing or the system. Any operation that “opens” the system is a potential site of infection. Closed system transfer is defined as the movement of sterile products from one container to another in which the container's closure system and transfer devices remain intact throughout the entire transfer process, compromised only by the penetration of a sterile, pyrogen-free needle or cannula through a designated closure or port to effect transfer, withdrawal, or delivery. A closed system transfer device can be effective but risk of infection is generally higher due to the mechanical closures typically used.

In the development of a glucose measurement system for frequent measurements in the intensive care unit, the ability to operate in a sterile or closed manner is extremely important. In the care of critically ill patients the desire to avoid the development of systemic or localized infections is considered extremely important. Therefore, any system that can operate in a completely closed manner without access to the peripheral environment is desired. For example, blood glucose measurement systems that require the removal of blood from the patient for glucose determination result in greater infection risk due to the fact that the system is exposed to a potentially non-sterile environment for each measurement. There are many techniques to minimize this risk of infection but the ideal approach is simply a system that is completely closed and sterilized. With respect to infection risk, a noncontact spectroscopic glucose measurement is almost ideal as the measurement is made with light which is able to evaluate the sample without any increase in infection risk.

### SUMMARY OF THE INVENTION

The present invention comprises methods and apparatuses that can provide measurement of glucose and other analytes with a variety of sensors without many of the performance-degrading problems of conventional approaches. An apparatus according to the present invention comprises a blood access system, adapted to remove blood from a body and infuse at least a portion of the removed blood back into the body. Such an apparatus also comprises an analyte sensor, mounted with the blood access system such that the analyte sensor measures the analyte in the blood that has been removed from the body by the blood access system. A method according to the present invention comprises removing blood from a body, using an analyte sensor to measure an analyte in the removed blood, and infusing at least a portion of the removed blood back into the body. The use of a non-contact sensor with a closed system creates a system will minimal infection risk. Advantages and novel features will become apparent to those skilled in the art upon examination of the following description or can be learned by practice of the invention. The advantages of the invention can be realized and attained by means of the methods, instrumentation architectures, and combinations specifically described in the disclosure and in the appended claims.

### BRIEF DESCRIPTION OF THE DRAWINGS

FIG. 1 is a scatter plot of 542 paired glucose measurements from “Experience with continuous glucose monitoring system a medical intensive care unit”, by Goldberg at al, Diabetes Technology and Therapeutics, Volume 6, Number 3, 2004.

FIG. 2 is an illustration of error grid analysis of glucose readings.

FIG. 3 is a schematic illustration of an example embodiment of the present invention comprising a blood access system using a blood flow loop.

FIG. 4 is a schematic illustration of a blood loop system with a peristaltic pump.

FIG. 5 is a schematic illustration of a blood access system implemented based upon a pull-push mechanism with a second circuit provided to prevent fluid overload.

FIG. 6 is a schematic illustration of a blood access system based upon a pull-push mechanism with a second circuit provided to prevent fluid overload.

FIG. 7 is a schematic illustration of a blood access system based upon a pull-push mechanism.

FIG. 8 is a schematic illustration of a blood access system implemented based upon a pull-push mechanism with a second circuit provided to prevent fluid overload.

FIG. 9 is a schematic illustration of an example embodiment that allows a blood sample for measurement to be isolated at a point near the patient and then transported to the instrument for measurement.

FIG. 10 is an illustration of the control of the blood volume and the integration of the total amount of glucose measured.

FIG. 11 is a schematic illustration of an example embodiment that allows a blood sample for measurement to be isolated at a point near the patient and then transported to the instrument for measurement through the use of leading and the following air gaps.

FIG. 12 is a schematic illustration of an example embodiment of the present invention.

FIG. 13 is a schematic illustration of an example embodiment of the present invention.

FIG. 14 is a schematic illustration of an example embodiment of the present invention.

FIG. 15 is a schematic illustration of an example embodiment of the present invention.

FIG. 16 is a plot showing the relationship between pressure, tubing diameter and blood fraction.

FIG. 17 is a plot showing the relationship between pressure, tubing diameter and blood fraction.

FIG. 18 is a schematic illustration of an example embodiment of the present invention.

FIG. 19 is a schematic illustration of an example embodiment of the present invention.

FIG. 20 is a schematic illustration of an example embodiment of the present invention.

FIG. 21 is a schematic illustration of the operation of an example embodiment of the present invention.

FIG. 22 is a schematic illustration of the operation of an example embodiment of the present invention,

FIG. 23 is a schematic illustration of an example embodiment of the present invention.

FIG. 24 is a schematic illustration of an example embodiment of the present invention.

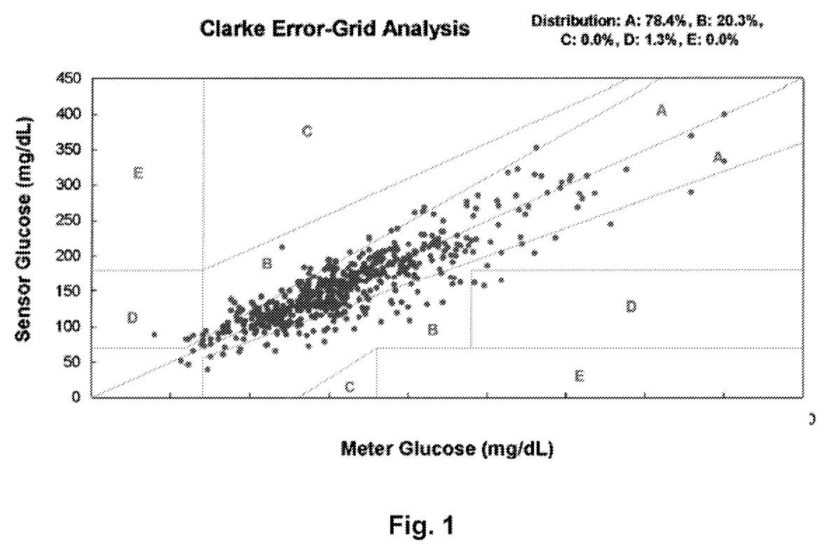

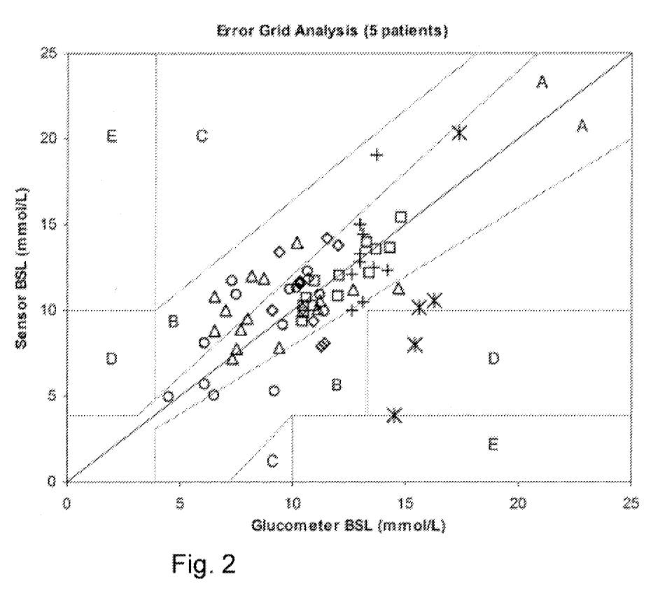

### DETAILED DESCRIPTION OF THE INVENTION

The present invention comprises methods and apparatuses that can provide measurement of glucose and other analytes with a variety of sensors without many of the performance-degrading problems of conventional approaches. An apparatus according to the present invention comprises a blood access system, adapted to remove blood from a body and infuse at least a portion of the removed blood back into the body. Such an apparatus also comprises an analyte sensor, mounted with the blood access system such that the analyte sensor measures the analyte in the blood that has been removed from the body by the blood access system. A method according to the present invention comprises removing blood from a body, using an analyte sensor to measure an analyte in the removed blood and infusing at least a portion of the removed blood back into the body.

The performance of the analyte sensor in the present invention can be dramatically improved compared with conventional applications by minimizing various issues that contribute to degraded sensor performance over time and by providing for cleaning and calibrating the measurement sensor over time. The physiological lag problems associated with conventional tissue measurements can also be reduced with the present invention by making a direct measurement in blood or by ensuring that there is appropriate agreement between the ISF glucose level and that in whole blood.

Some embodiments of the present invention provide for effective cleaning of the sensor. If effectively cleaned at the end of each measurement, the amount of sensor fouling and/or drift can be minimized. Saline or another physiologically compatible solution can be used to clean the sensing element.

A typical glucose sensor used relies on a glucose-dependent reaction to measure the amount of glucose present. The reaction typically uses both oxygen and glucose as reactants. If either oxygen or glucose is not present, the reaction can not proceed some embodiments of the present invention provide for total removal of one or the other to allow a zero point calibration condition. Saline or another physiological compatible solution that does not contain glucose could be used to effectively create a zero point calibration condition.

There can be limitations associated with a zero point calibration so that one may desire to use a calibration point with a glucose value above zero and preferably within the physiological range. Some embodiments of the present invention provide for such a calibration by exposing the sensor to a glucose containing solution with a known glucose concentration. This can effectively recalibrate the sensor and improve its accuracy. The ability to make frequent recalibrations enables a simplistic approach to maintaining overall sensor accuracy.

In many medical laboratory measurement products a two point calibration is used. Some embodiments of the present invention provide two types of calibrations to provide a two point calibration capability. A two point calibration can allow both bias and slope to be effectively determined and mitigated.

In practice the degree or amount of physiological lag observed between ISF glucose levels in whole blood glucose levels creates a significant error source. Some embodiments of the present invention reduce this source of error by placing the sensor in direct contact with blood.

Recognizing the several error sources, the present invention provides an accurate continuous or semicontinuous blood glucose measurement system for use in applications such as the intensive care unit. Some embodiments of the present invention place blood in contact with a sensing mechanism for a defined measurement period and then clean the sensor. Following cleaning of the sensor, a calibration point or points can be established. The present invention contemplates a variety of blood access circuits that can enable the sensor to be cleaned on a periodic basis and can allow for recalibration; illustrative examples are described below. In addition to providing a mechanism for improved sensor performance, the disclosed blood access systems can also provide methods for occlusion management, minimization of blood loss and minimization of saline used for circuit cleaning.

The example embodiments generally show a blood access system with the ability to control fluid flows at a location removed from the blood access console and near the patient. The ability to control fluid flows at this remote location does not necessitate the use of a mechanical valve or other similar apparatus that similarly directs or control flow at a point near the patient. Additionally it does not require nurse or other human intervention. For multiple reasons, including safety and reliability, it is desirable not to have a mechanical device, wires, or electrical power near the patient. As shown in many example embodiments, this capacity is enabled through the use of a pumping mechanism that provides for both fluid stoppage and movement. Additional capabilities are provided by bidirectional operation of the pumps, and by operation at variable speeds including complete stoppage of fluid flow fluid flow. As used in the disclosure, operation may be the use of the pump as a flow control device to prevent flow. As shown in the example embodiments these capabilities can be provided through peristaltic pumps and syringe pumps. It is recognized by one of ordinary skill in the art that these capabilities can also be provided by other fluid handling devices, including as examples linear “finger” pumps, valveless rotating and reciprocating piston metering pumps, piston pumps, lifting pumps, diaphragm pumps, and centrifugal pumps. “Plunger” pumps to include syringe pumps as well as those that can clean a long thin flexible piece of tubing are considered. These types of plunger pumps have the advantage of removing or transporting the fluid without the need for a following fluid volume. For example, no follow volume is required when using a syringe pump.

The example embodiments generally show a sensor in contact with a blood access system. The sensor can be immersed or otherwise continuously exposed to fluid in the system. It can also comprise a noncontact sensor that interacts with fluid in the system. It can also comprise a sensor remote from the blood access system, where the sensor element in the example comprises a port or other sampling mechanism that allows a suitable sample of fluid from the system to be extracted and presented to the remote sensor. This type of sampling can be used with existing technology glucose meters and reagent strips.

Example Embodiment Comprising a Sensor and a Fluid Management System.

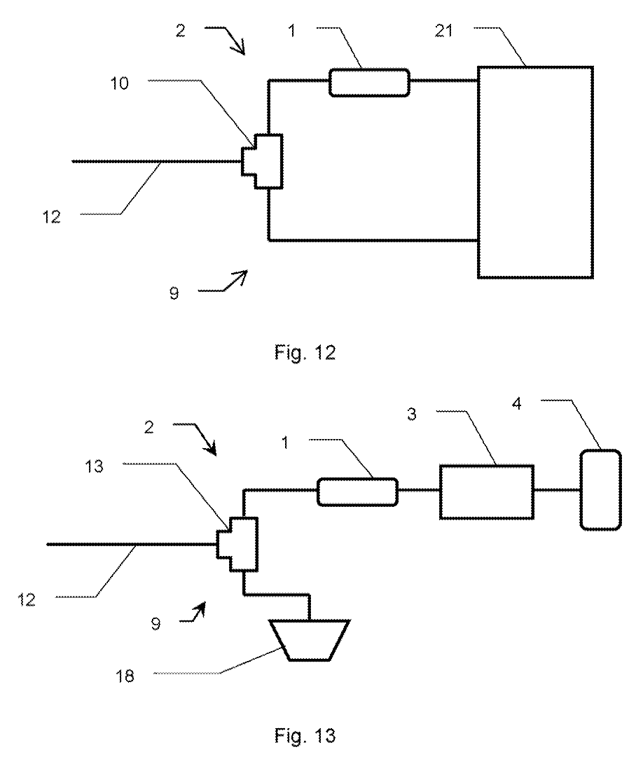

FIG. 12 is a schematic illustration of an example embodiment of the present invention comprising a sensor and a fluid management system. The system comprises a catheter (or similar blood access device) (12) in fluid communication with the vascular system of a patient. A tubing extension (if required) extends from the catheter (12) to a junction (10). A first side of the junction (10) connects with fluid transport apparatus (2) such as tubing (for reference purposes called the “left side” of the blood system); a second side of the junction (10) connects with fluid transport apparatus (9) such as tubing (for reference purposes called the “right side” of the blood system). A sensor (1) mounts with the left side (2) of the blood loop. A fluid management system (21) is in fluid communication with the left side (2) and right side (9) of the blood system. In operation, the fluid management system (21) acts to draw blood from the patient through the catheter 12 and into the left side (2) of the blood system to the sensor 1. The sensor 1 determines a blood property of interest, for example the concentration of glucose in the blood. The fluid management system (21) can push the blood back to the patient through the left side (2) of the blood system, or can further draw the measured blood into the right side (9) of the blood system, and through junction (10) to catheter (12) and back into the patient.

The fluid management system (21) can control the fluid volume flow and fluid pressure in the left (2) and right (9) sides of the blood system to control whether fluid is being withdrawn from the patient, infused into the patient, or neither. The fluid management system (21) can also comprise a source of a suitable fluid such as saline, and manage fluid flow in the system such that saline is circulated through the left (2) and right (9) sides to flush or clean the system. The fluid management system can further comprise an outlet to a waste container or channel, and manage fluid flow such that used saline, blood/saline mix, or blood that is not desired to be returned to the patient (depending on the requirements of the application) is delivered to the waste container or channel.

Example Embodiment Comprising a Blood Loop System with a Syringe Pump.

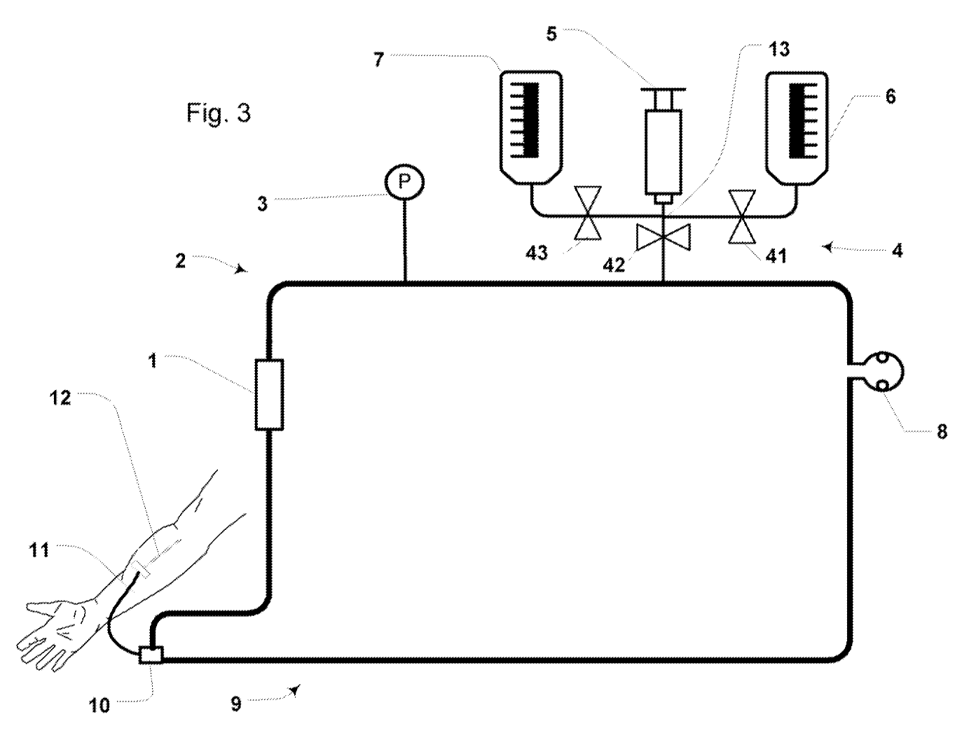

FIG. 3 is a schematic illustration of an example embodiment of the present invention comprising a blood access system using a blood flow loop. The system comprises a catheter (or similar blood access device) (12) in fluid communication with the vascular system of a patient. A tubing extension (11) (if required) extends from the catheter (12) to a junction (10). A first side of the junction (10) connects with fluid transport apparatus (2) such as tubing (for reference purposes called the “left side” of the blood loop); a second side of the junction (10) connects with fluid transport apparatus (9) such as tubing (for reference purposes called the “right side of the blood loop). A sensor measurement cell (1) and a pressure measurement device (3) mount with the left side (2) of the blood loop. A peristaltic pump (8) mounts between the left side (2) and the right side (9) of the blood loop. A pinch valve (42) (“pinch valve” is used for convenience throughout the description to refer to a pinch valve or any suitable flow control mechanism) mounts between the left side (2) of the blood loop and a junction (13) controlling fluid communication therebetween. A pinch valve (43) mounts between the junction (13) and a waste channel (7) (such as a bag), controlling fluid communication therebetween. A pinch valve 41 mounts between the junction(13) and a source of wash fluid (6) (such as a bag of saline)>controlling fluid flow therebetween. A syringe pump (5) mounts in fluid communication with the junction (13). The system can be operated as described below. The description assumes a primed state of the system wherein saline or another appropriate fluid is used to initially fill some or all channels of fluid communication. Those skilled in the art will appreciate that other start conditions are possible. Note that “left side” and “right side are for convenience of reference only, and are not intended to limit the placement or disposition of the blood loops to specific left-right relationship.

Blood sample and measurement process. A first sample draw with the example embodiment of FIG. 3 can be accomplished with the following steps:
1. Syringe pump (5) initiates a draw along the left side (2) of the blood loop.
2. The blood interacts with the sensor measurement cell (1). The volume of the catheter (12) and extension tubing (11) can be determined from the syringe pump (5) operating parameters and the time until blood is detected by the sensor measurement cell (1) and used for future reference.
3. Sensor measurements can be made as the blood moves through the measurement cell (1).
4. As blood nears junction (13) the system can be stopped and the saline that was drawn into the syringe pump (5) placed in waste bag (7) by the appropriate use of pinch valves (43, 42, 41).
5. Blood drawn via the left side can continue via the withdrawal of syringe (5).
6. Withdrawal of blood by the syringe either Fully or partially, is stopped. Sensor sampling of the measurement cell can be continued or stopped.
7. Initially saline and then blood is re-infused into the subject via combination of peristaltic pump (8) and syringe (5). The two pump mechanisms operate at the same rate such that blood is moved along the right side (9) of the circuit only. Note, blood does not substantially progress up the left side (2) of the circuit but is re-infused past junction (10) and into the patient.
8. One or more weight scales (not shown) can be used to measure the waste and saline solution together or independently. Such weight scales can allow real time compensation between the pumps, e.g., to ensure that the rates match, or to ensure that a desired rate difference or bias is maintained. For instance it can be desirable that a certain volume of saline be infused into the patient during a recirculation cycle. In such an application, the combined weight of the waste and saline bag should decrease by the weight of the desired volume of saline. If the weight or weights do not correspond to the expected weight or weights, then one or both pumps can be adjusted. If a net zero balance is required then the combined weight at the start of recirculation mode and at the end of recirculation mode should be the same; again, one or both pumps can be adjusted to reach the desired weight or weights.

Subsequent Blood Sampling. For subsequent samples, the blood residing in the catheter (12) and extension tubing (11) has already been tested and can be considered a “used,” sample. The example embodiment of FIG. 3 can prevent this sample from contaminating the next measurement, by operation as follows.
1. Syringe pump (5) and peristaltic pump (8) initiate the blood draw by drawing blood up through the right side of the blood loop.
2. The withdrawal continues until all of the used blood has passed junction (10). The volume determination made during the initial draw can enable the accurate determination of the location of the used blood sample.
3. Once the used sample has passed the junction (10), the peristaltic pump (8) can be turned off and blood withdrawn via the left side (2) of the circuit. Sensor measurement of the blood can be made during this withdrawal.
4. The withdrawal process can continue for a predetermined amount of time. Following completion of the sensor sampling (or overlapped in time), the blood can be re-infused into the patient. The blood is re-infused into subject via combination of peristaltic pump (8) and syringe pump (5). The two pumps operate at the same rate such that blood is moved along the right side (9) of the circuit only. Note, blood does not progress up the left side (2) of the circuit but is re-infused past junction (10) and into the patient. There is no requirement that the withdrawal and infusion rates be the same for this blood loop system.

Cleaning of system and saline calibration procurement. A cleaning and calibration step can clean the system of any residual protein or blood build-up, and can characterize the system, e.g., the performance of a measurement system can be characterized by making a saline calibration reference measurement, and that characterization used in error reporting, instrument self-tests, and to enhance the accuracy of blood measurements. The cleaning process can be initiated at the end of a standard blood sampling cycle, at the end of each cycle, or at the end of each set of a predetermined number of cycles, at the end of a predetermined time, when some performance characterization indicates that cleaning is required, or some combination thereof. A cleaning cycle can be provided with the example embodiment of FIG. 3 with a method such as the following.
1. The start condition for initiation of the cleaning cycle has the syringe substantially depressed following infusion of blood into the patient.
2. Pinch valve (42) closed and pinch valve (41) opened and syringe (5) withdraws saline from the wash bag (6).
3. Following the withdrawal, pinch valve (42) is opened and (41) and (43) are closed.
4. Syringe pump (5) pushes saline toward patient at first rate while peristaltic pump (8) operates at a second rate equal to one half of the first rate. This rate relationship means that saline is infused into the two arms for the loop at equal rates and the blood present in the system is re-infused into the patient.
5. Following completion of the saline infusion, both arms of the loop system (2, 9) as well as the tubing (11) and catheter (12) are filled with saline.
6. Pinch valve (42) is dosed and peristaltic pump (8) is turned on in a vibrate mode or pulsatile flow mode to completely clean the loop.
7. Pinch value (42) is opened. Syringe begins pull at a third rate and peristaltic pump pulls saline at fourth rate equal to one half of the third rate. This process effectively fills the entire loop with blood while concurrently placing the saline used for cleaning into the syringe (5). Sensor measurements can occur after the blood/saline junction has passed the measurement cell.
8. Pinch valve (43) opened and pinch valve (42) closed and saline is infused into waste bag (7).
9. Pinch valve (43) closed, (42) opened and blood pulled from patient and back to measurement mode.

Characteristics of the example embodiment. The example embodiment of FIG. 3 allows sensor measurements of blood to be made on a very frequent basis in a semi-continuous fashion. There is little or no blood loss except during the cleaning cycle. Saline is infused into the patient only during cleaning, and very little saline is infused into the patient. The gas dynamics of the system can be fully equilibrated, allowing the example embodiment to be used with arterial blood. There are no blood/saline junction complications except during cleaning. The system contains a pressure monitor that can provide arterial, central venous, or pulmonary artery catheter pressure measurements after compensation for the pull and push of the blood access system. The system can compensate for different size catheters through the volume pulled via the syringe pump. The system can determine occlusions or partial occlusions with the blood sensor or the pressure sensor. Due to the flexibility in operation and the direction of flow, the system can determine if the occlusion or partial occlusion is in the left side of the circuit, the right side of the circuit or in the tubing between the patient and the T-junction. If the occlusion is in the right or left sides, the system can enter a cleaning cycle with agitation and remove the clot build-up. If a microembolus is detected the system can initiate a mode of operation such that the problematic blood is taken directly to waste. The system can then enter into a mode such that it becomes saline filled but does not initiate additional blood withdrawals. In the case of microemboli detection, the system has effectively managed the potentially dangerous situation and the nurse can be notified to examine the system for emboli formation centers such as poorly fitting catheter junctions.

Example Embodiment Comprising a Blood Loop System with a Peristaltic Pump.

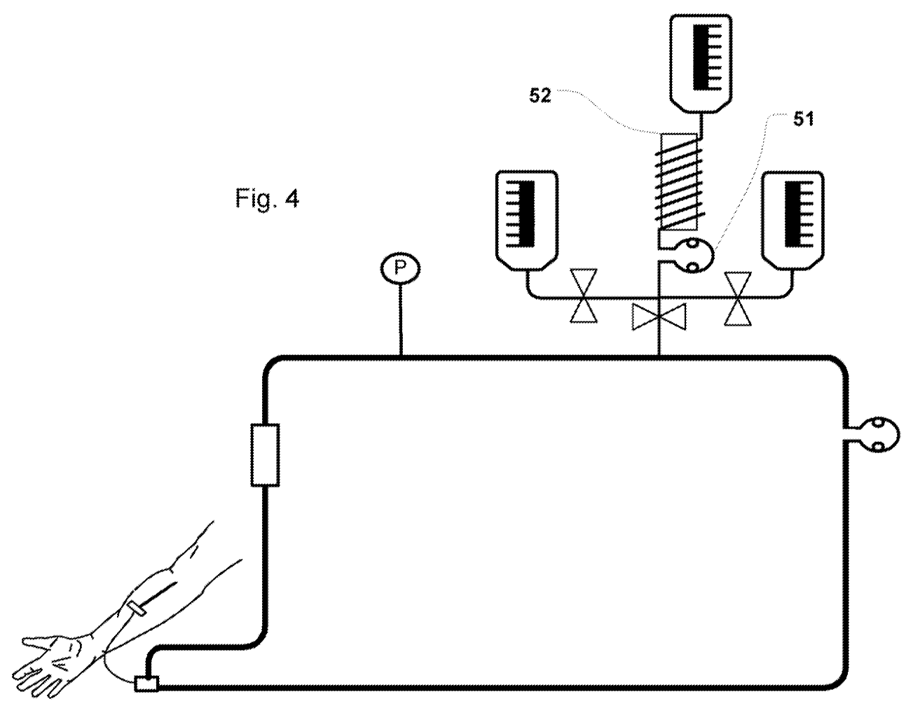

FIG. 4 is a schematic illustration of a blood loop system with a peristaltic pump. The system of FIG. 4 is similar to that of FIG. 3, with the syringe pump of FIG. 3 replaced by a peristaltic pump (51) and a tubing reservoir (52). The reservoir as used in this application is defined as any device that allows for the storage of fluid. Examples included are a piece of tubing, a coil of tubing, a bag, a flexible pillow, a syringe, a bellows device, or any device that can be expanded through pressure, a fluid column, etc. The operation of the system is essentially unchanged except for variations that reflect the change from a syringe pump to a peristaltic or other type of pump. The blood loss and saline consumption requirements of the system are of course different due to the blood saline interface present in the operation of the second peristaltic pump. Unlike the syringe pump of FIG. 3, the example embodiment of FIG. 4 must maintain a sterile compartment and minimize the contact between air and blood for many applications. A saline fluid column can fill the tubing, and effectively moves up and down as fluid is with drawn by the peristaltic pump.

Push Pull System.

FIG. 13 is a schematic illustration of a blood access system according to the present invention. The system comprises a catheter (or similar blood access device) (12) in fluid communication with the vascular system of a patient. A tubing extension (if required) extends from the catheter (12) to a junction (13). A first side of the junction (13) connects with fluid transport apparatus (2) such as tubing (for reference purposes called the “left side” of the blood system); a second side of the junction (13) connects with fluid transport apparatus (9) such as tubing (for reference purposes called the “right side” of the blood system). A sensor (1) is in fluid communication with the left side (2) of the system. A pump (3) is in fluid communication with the left side (2) of the system (shown in the figure as distal from the patient relative to the sensor (1); the relative positions can be reversed). A source (4) of suitable fluid such as saline is in fluid communication with the left side (2) of the system. A waste container (18) or connection to a waste channel is in fluid communication with the right side (9) of the system. In operation, the pump (3) operates to draw blood from the patient through the catheter (12) and junction (1 3) into the left side (2) of the system. The sensor (1) determines a desired property of the blood, e.g., the glucose concentration in the blood. The pump (3) operates to draw saline from the container (4) and push the blood back into the patient through junction (13) and catheter (12). After a sufficient quantity of blood has been re-infused (e.g., by volume, or by acceptable blood/saline mixing threshold), then the pump (3) operates to push remaining blood, blood/saline mix, or saline into the right side (9) of the system and into the waste container (18) or channel. The transport of fluid from the left side (2) to the right side (9) of the system can be used to clear undesirable fluids (e.g., blood/saline mixtures that are not suitable for re-infusion or measurement) and to flush the system to help in future measurement accuracy. Valves, pumps, or additional flow control devices can be used to control whether fluid from the left side (2) is infused into the patient or transported to the right side (9) of the system; and to prevent fluid from the right side (9) of the system from contaminating blood being withdrawn into the left side (2) of the system for measurement.

Push Pull System with Two Peristaltic Pumps.

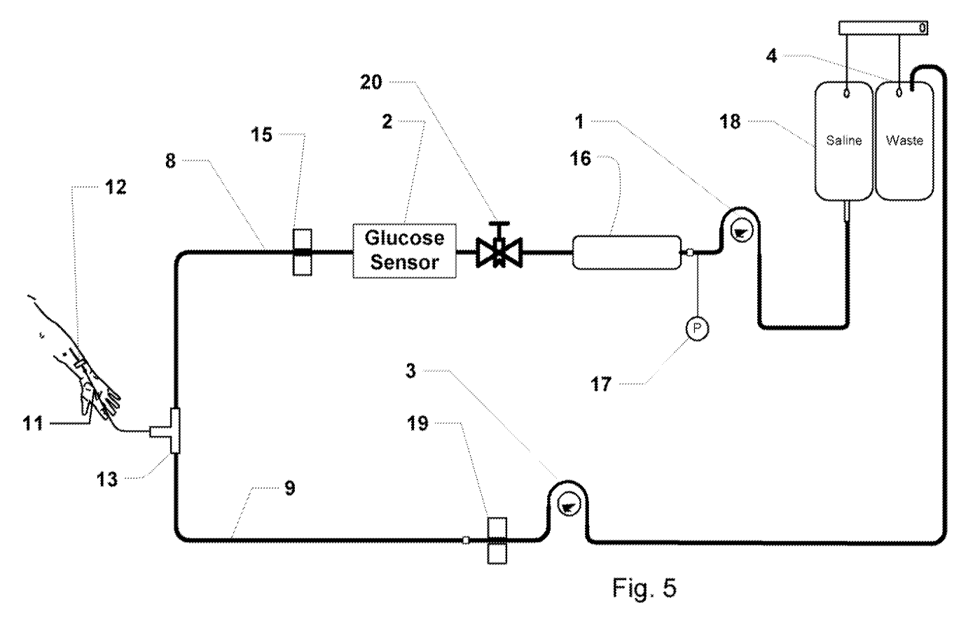

FIG. 5 is a schematic illustration of a blood access system implemented based upon a pull-push mechanism with a second circuit provided to prevent fluid overload of the patient. The system comprises a catheter (or similar blood access device) (12) in fluid communication with the vascular system of a patient. A tubing extension (11) (if required) extends from the catheter (12) to a junction (13). A first side of the junction (13) connects with fluid transport apparatus (8) such as tubing (for reference purposes called the “left side” of the blood loop); a second side of the junction (13) connects with fluid transport apparatus (9) such as tubing (for reference purposes called the “right side” of the blood loop). An air detector (1 5) that can serve as a leak detector, a pressure measurement device (17), a glucose sensor (2), and a needle-less blood access port (20) mount with the left side of the blood loop. A tubing reservoir (16) mounts with the left side of the blood loop, and is in fluid communication with a blood pump (1). Blood pump (1) is in fluid communication with a reservoir (18) of fluid such as saline. A blood leak detector (19) serves as a safety that can serve as a leak detector mounts with the right side of the blood loop. A second blood pump (3) mounts with the right side of the blood loop, and is in fluid communication with a receptacle or channel for waste, depicted in the figure as a bag (4). Elements of the system and their operation are further described below.

Blood Sample and Measurement Process—First Sample Draw.
1. Pump (1) initiates a draw of blood from the catheter (12).
2. The blood interacts with the sensor measurement cell (2). The volume of the catheter (12) and tubing (11) can be determined and used for future reference and for the determination of blood-saline mixing.
3. Sensor measurements can be made as the blood moves through the measurement cell.
4. Pump (1) changes direction and sensor measurements continue.
5. Pump (1) re-infuses blood into the patient. As the mixed blood-saline junction passes the junction (13), it becomes progressively more dilute.
6. Following re-infusion of the majority of the blood, peristaltic pump (3) is turned on and the saline with a small amount of residual blood is taken to the waste bag (4).
7. The system can be washed with saline after each measurement if desired.
8. Additionally the system can go into an agitation mode that fully washes the system
9. Finally the system can enter into a keep vein open mode (KVO). In this mode a small amount of saline is continuously or periodically infused to keep the blood access point open.

Blood sample and measurement process—Subsequent Blood Sampling. For subsequent samples, the tubing between the patient and the pump (1) is filled with saline and it can be desirable that this saline not become mixed with the blood. This can be achieved with operation as follows:
1. Pump (1) initiates the blood draw by drawing blood up through junction(13).
2. The withdrawal continues as blood passes through the sensor measurement cell (2). The blood after passing the measurement cell can be effectively stored in the tubing reservoir (5).
3. Sensor measurements can be made during this withdrawal period.
4. Following completion of the blood withdrawal, the blood can be re-infused into the patient by reversing the direction of pump (1).
5. Sensor measurements can also be made during the re-infusion period. 6b As the mixed blood-saline passes through the junction (13), it becomes progressively more dilute.
7. Following re-infusion of the majority of the blood peristaltic pump (3) is turned on at a rate that matches the rate of pump (1). The small amount of residual blood mixed with the saline is taken to the waste bag (4).
8. This process results in a washing of the system with saline,
9. Additional system cleaning is possible through an agitation mode. In this mode the fluid is moved forward and back such that turbulence in the flow occurs.
10. Between blood samplings, the system can be placed in a keep vein open mode (KVO). In this mode a small amount of saline can be infused to keep the blood access point open.

Characteristics of Push Pull with Peristaltic Pumps. The example embodiment of FIG. 5 can operate with minimal blood loss since the majority of the blood removed can be returned to the patient. The diversion of saline into a waste channel can prevent the infusion of significant amounts of saline into the patient. The pump can be used to compensate for different sizes of catheters. The system can detect partial or complete occlusion with either the analyte sensor or use of pressure sensor (17) or additional pressure sensors not shown. An occlusion can be cleared through a variety of means. For example if the vein is collapsing and the system needs to re-infuse saline either the blood pump or the flush pump can be used to effectively refill the vein. If there is evidence of occlusion in the measurement cell area, the both the blood pump and flush pumps can be activated such that significant fluid can be flushed through the system for effective cleaning. In addition to high flow rates the bidirectional pump capabilities of the pumps can be used to remove occlusions. If a microembolus is detected the system can initiate a mode of operation such that the problematic blood is taken directly to waste. The system can then enter into a mode such that it becomes saline filled but does not initiate additional blood withdrawals. In the case of microemboli detection, the system has effectively managed the potentially dangerous situation and the nurse can be notified to examine the system for emboli formation centers such as poorly fitting catheter junctions.

Push Pull System with Syringe Pump.

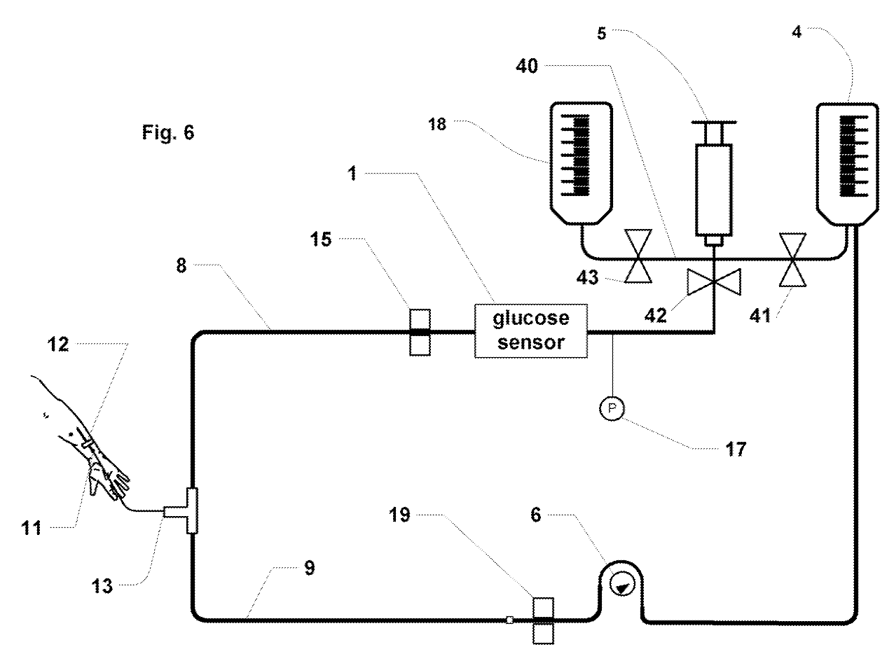

FIG. 6 is a schematic illustration of a blood access system based upon a pull-push mechanism with a second circuit provided to prevent fluid overload of the patient. The system comprises a catheter (or similar blood access device) (12) in fluid communication with the vascular system of a patient. A tubing extension (11) (if required) extends from the catheter (12) to a junction (13). A first side of the junction (13) connects with fluid transport apparatus (8) such as tubing (for reference purposes called the “left side” of the blood loop)y a second side of the junction (13) connects with fluid transport apparatus (9) such as tubing (for reference purposes called the “right side” of the blood loop). An air detector (15) that can serve as a leak detector, a pressure measurement device (17), and a glucose sensor (1) mount with the left side of the blood loop. A pinch valve (42) mounts between the left side (2) of the blood loop and a junction (40), controlling fluid communication therebetween. A pinch valve (41) mounts between the junction (40) and a waste channel (4) (such as a bag), controlling fluid communication therebetween. A pinch valve (43) mounts between the junction (40) and a source of wash fluid (18) (such as a bag of saline), controlling fluid flow there between. A syringe pump (5) mounts in fluid communication with the junction (40). A blood leak detector (19) that can serve as a leak detector mounts with the right side of the blood loop. A second blood pump (6) mounts with the right side of the blood loop, and is in fluid communication with a receptacle or channel for waste, depicted in the figure as a bag (4). Elements of the system and their operation are further described below.

Blood Sample and Measurement Process—First Sample Draw.
1. Syringe pump (5) initiates a draw.
2. The blood interacts with the sensor measurement cell (1). The volume of the catheter (12) and tubing (11) can be determined and used for future reference and for the determination of blood-saline mixing.
3. Sensor measurements can be made as the blood moves through the measurement cell.
4. The syringe pump changes direction and sensor measurements can continue.
5. Blood is re-infused into the patient. As the mixed blood-saline junction passes the junction (13), it becomes progressively more dilute.
6. Following re-infusion of a portion (e.g., the majority) of the blood, peristaltic pump (6) is turned on and the saline with a small amount of residual blood is taken to the waste bag.
7. The system can be washed with saline after each measurement if desired.
8. Additionally the system can go into an agitation mode that fully washes the system.
9. Finally the system can enter a keep vein open mode (KVO). In this mode a small amount of saline is infused to keep the blood access point open.

Blood sample and measurement process—Subsequent Blood Sampling. For subsequent samples, the tubing between the patient and the syringe is filled saline and it can be desirable that this saline not become mixed with the blood. The pinch valves enable the saline to be pushed to waste and the amount of saline/blood mixing to be minimized. This can be achieved with operation as described below.
1. Syringe pump (5) initiates the blood draw by drawing blood up through junction (13).
2. The withdrawal continues until blood saline juncture reaches the base of the syringe. At this point in the sequence, pinch valve (42) is closed and valve (41) is opened, and the syringe pump direction reversed. This process enables the resident saline to be placed into the waste bag.
3. Valve (42) is opened, valve (41) closed and the syringe is now withdrawn so that only blood or blood with very little saline contamination is pulled into the syringe.
4. Sensor measurements can be made during this withdrawal period.
5. Following completion of the blood withdrawal, the blood is re-infused into the patient by reversing the direction of the syringe pump. As the mixed blood-saline passed through the junction (13), it becomes progressively more dilute.
6. Following re-infusion of the majority of the blood, peristaltic pump (6) is activated with the concurrent infusion from the syringe pump and the saline with a small amount of residual blood it taken to the waste bag.
7. This process results in a washing of the system with saline.
8. Additional system cleaning is possible through an agitation mode. In this mode the fluid is moved forward and back such that turbulence in the flow occurs.
9. Between blood samplings, the system can be placed in a keep vein open mode (KVO). In this mode a small amount of saline is infused to keep the blood access point open.

Characteristics of Push Pull with Syringe Pump. The system can operate with little blood loss since the majority of blood is re-infused into the patient. The diversion of saline to waste can result in very little saline infused into the patient. Saline mixing occurs only during blood infusion. The pressure monitor can provide arterial, central venous, or pulmonary artery catheter pressure measurements after compensation for the pull and push of the blood access system. The system can compensate for different size catheters through the volume pulled via the syringe pump,

The system can detect partial or complete occlusion with either the analyte sensor or the pressure sensor. An occlusion can be cleared through a variety of means. For example if the vein is collapsing and the system needs to re-infuse saline either the syringe pump or the flush pump can be used to effectively refill the vein. If there is evidence of occlusion in the measurement cell area, both the syringe pump and flush pumps can be activated such that significant fluid can be flushed through the system for effective cleaning. In addition to high flow rates the bidirectional pump capabilities of the pumps can be used to remove occlusions.

The syringe pump mechanism can also have a source of heparin or other anticoagulant attached through an additional port (not shown). The anticoagulant solution can then be drawn into the syringe and infused into the patient or pulled through the flush side of the system. The ability to rinse the system with such a solution can be advantageous when any type of occlusion is detected.

If a microembolus is detected the system can initiate a mode of operation such that the problematic blood is taken directly to waste. The system can then enter into a mode such that it becomes saline filled but does not initiate additional blood withdrawals. In the case of microemboli detection the system has effectively managed the potentially dangerous situation and the nurse can be notified to examine the system for emboli formation centers such as poorly fitting catheter junctions.

Push Pull System with Syringe & Peristaltic Pump.

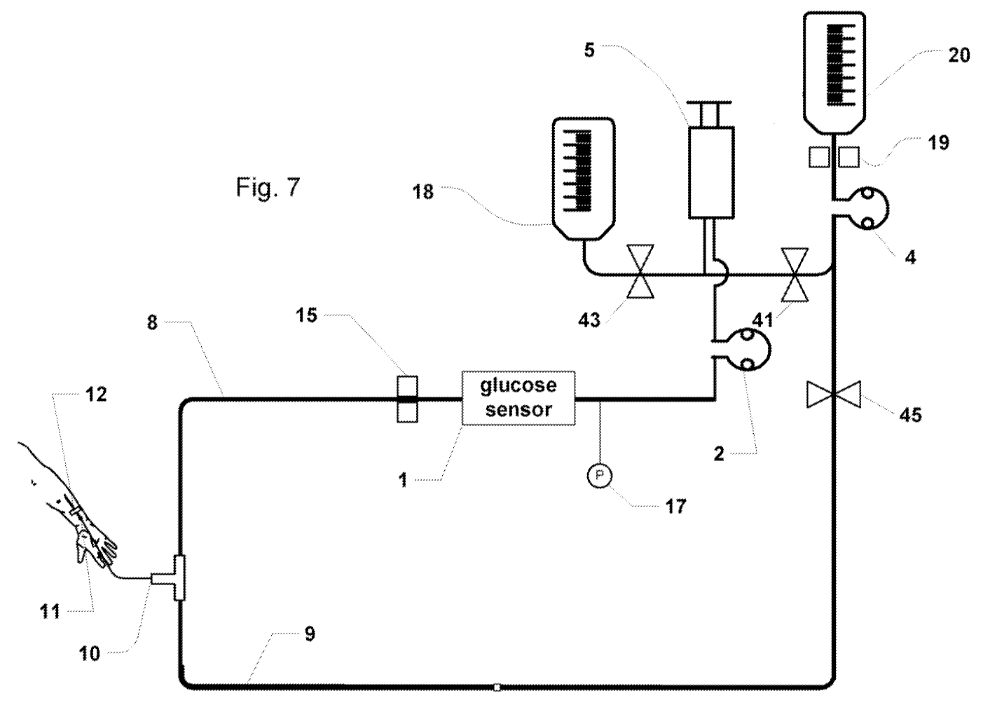

FIG. 7 is a schematic illustration of another example push pull system. The system comprises a catheter (or similar blood access device) (12) in fluid communication with the vascular system of a patient. A tubing extension (11) (if required) extends from the catheter (12) to a junction (10). A first side of the junction (10) connects with fluid transport apparatus (8) such as tubing (for reference purposes called the “left side” of the blood loop); a second side of the junction (10) connects with fluid transport apparatus (9) such as tubing (for reference purposes called the “right side” of the blood loop), An air detector (15) that can serve as a leak detector, a pressure measurement device (17), and a glucose sensor (1) mount with the left side of the blood loop. A blood pump (2) mounts with the left side of the blood loop such that it controls flow between a passive reservoir (5) and the left side of the blood loop. A pinch valve (45) mounts with the right side of the blood loop: controlling flow between the right side of the blood loop and a second pump (4). The second pump (4) is also in fluid communication with a waste channel such as a bag (20): with a leak detector (19) mounted between the pump (4) and the bag (2). A pinch valve (41) mounts between the pump (4) and a port of the passive reservoir (5): which port is also in fluid communication with a pinch valve (43) between the port and a source of saline such as a bag (18). Elements of the system and their operation are further described below.

Blood Sample and Measurement Process—Sampling Process.
1. The passive reservoir is not filled and valve (41) is open,
2. Peristaltic pump (4) & pump (2) initiate the blood draw. The saline in the line moves into the saline bag.
3. As the blood approaches the syringe, pump (4) stops and valve (41) closes. The blood now moves into the passive reservoir.
4. Sensor sampling of the blood occurs in sensor (1).
5. Pump (2) reverses direction and the blood is infused into the patient.
6. The reservoir goes to minimum volume, at which point valve (43) opens and saline washes the reservoir and is used to push the blood back to the patient.
7. As the mixed blood-saline passes through the junction (13), it becomes progressively more dilute.
8. Following re-infusion of the majority of the blood or all of the blood, peristaltic pump (4) is turned on at the same rate as pump (2) and valves (45) and (43) are open. The combination of pumps creates a wash circuit that cleans the system.
9. Further washing of the syringe reservoir can occur by opening valves (43: 41) with pump (4) active,
10. Keep vein open infusions can occur by having pump (2) active with valve (43) open.

Characteristics of the Push Pull System with Syringe and Peristaltic Pump. Blood is always moving either into or out of the access system. Circuit cleaning can be independent of syringe cleaning. Blood loss is zero or minimal since the majority of blood is re-infused in to the patient. Very little saline is infused due to diversion of saline into waste and the fact that the mixing period is only during infusion. Saline mixing during blood infusion only. The system contains a pressure monitor that can provide arterial, central venous! or pulmonary artery catheter pressure measurements after compensation for the pull and push of the blood access system. The system can compensate for different size catheters through the volume pulled via the syringe pump.

The system can detect partial or complete occlusion with either the analyte sensor or the pressure sensor. An occlusion can be cleared through a variety of means. For example if the vein is collapsing and the system needs to re-infuse saline via either syringe pump, if there is evidence of occlusion in the measurement cell area, the both syringe pumps can be activated such that significant fluid can be flushed through the system for effective cleaning. In addition to high flow rates the bidirectional pump capabilities of the pumps can be used to remove occlusions. The flexibility of the described system with the various pinch valves allows one to identify the occlusion location and establish a proactive cleaning program to minimize further occlusion.

The syringe pump mechanism can also have a source of heparin or other anticoagulant attached through an additional port (not shown). The anticoagulant solution can then be drawn into the syringe and infused into the patient or pulled through the flush side of the system. The ability to rinse the system with such a solution could be advantageous when any type of occlusion is detected.

Push Pull System.

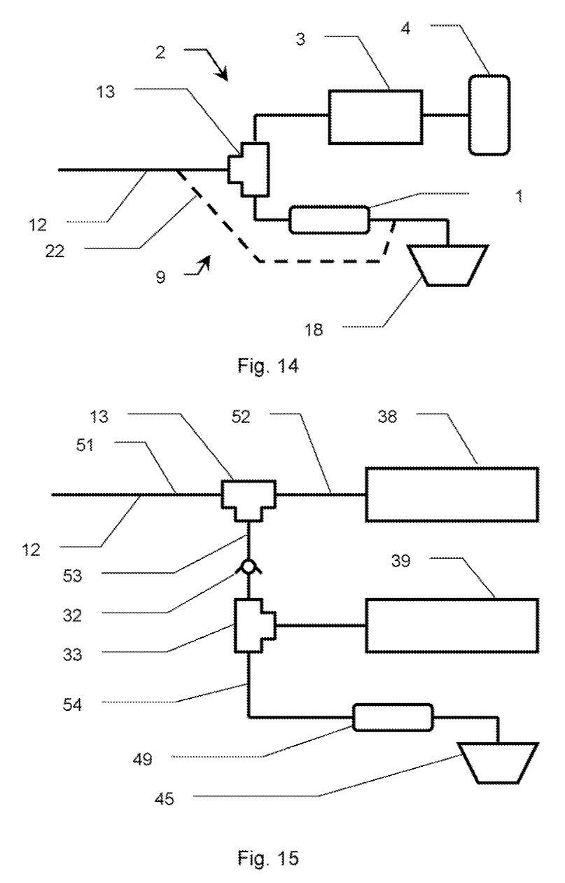

FIG. 14 is a schematic illustration of a blood access system according to the present invention. The system comprises a catheter (or similar blood access device) (12) in fluid communication with the vascular system of a patient. A tubing extension (if required) extends from the catheter (12) to a junction (13). A first side of the junction (13) connects with fluid transport apparatus (2) such as tubing (for reference purposes called the “left side” of the blood system); a second side of the junction (13) connects with fluid transport apparatus (9) such as tubing (for reference purposes called the “right side” of the blood system). A pump (3) is in fluid communication with the left side (2) of the system. A source (4) of suitable fluid such as saline is in fluid communication with the left side (2) of the system. A sensor (1) is in fluid communication with the right side (9) of the system. A waste container (18) or connection to a waste channel is in fluid communication with the right side (9) of the system. An optional fluid transport apparatus 22 is in fluid communication with the right side (9) of the system between the sensor (1) and the waste container (18) or channel, and with the patient (e.g., via the catheter (12)).

In operation, the pump (3) operates to draw blood from the patient through the catheter (12) and junction (13) into the left side (2) of the system. Once a sufficient volume of blood has been drawn into the left side (2), the pump operates to push the blood from the left side (2) to the right side (9), wherein the sensor (1) determines a desired blood property (e.g., the concentration of glucose in the blood). The pump (3) can draw saline from the bag (4) to push the blood through the system. Blood from the sensor (1) can be pushed to the waste container (18) or channel, or can optionally be returned to the patient via the optional return path (22). The transport of fluid through from the left side (2) to the right side (9) of the system can be used to clear undesirable fluids (e.g., blood/saline mixtures that are not suitable for re-infusion or measurement) and to flush the system to help in future measurement accuracy. Valves, pumps, or additional flow control devices can be used to control whether fluid is drawn from patient into the left side (2) or transported to the right side (9) of the system; and to prevent blood/saline mix and saline from the left side (9) of the system from being infused into the patient.

Push Pull with Additional Path.

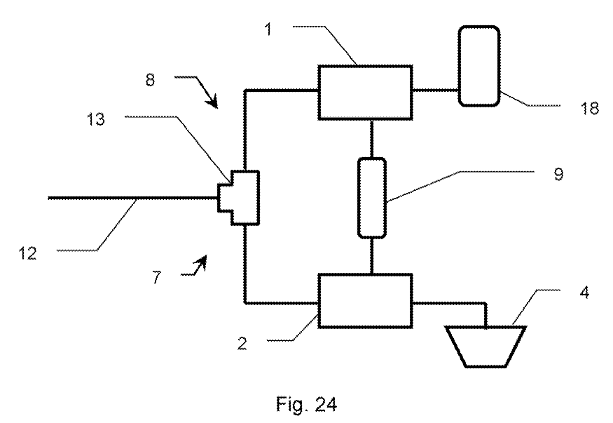

FIG. 24 is a schematic illustration of an example embodiment. The system comprises a catheter (or similar blood access device) (12) in fluid communication with the vascular system of a patient, and in fluid communication with a junction (13). A first side of the junction (13) connects with fluid transport apparatus (8) such as tubing (for reference purposes called the “left side” of the system). The left side of the system further comprises a source of maintenance fluid (18) and a connection to one side of a flow through glucose sensor system (9). A first fluid control system (1) controls fluid flow within the left side of the system. A second side of the junction (13) connects with fluid transport apparatus (7) such as tubing (for reference purposes called the “right side” of the system). The right side of the system further comprises a channel or receptacle for waste (4), and a connection to a second side of the flow through glucose sensor system (9). A second fluid control system (2) controls fluid flow within the left side of the system. In operation, the first and second fluid control systems are operated to draw blood from the patient to the junction (13), and then into either the left or right side of the system. The fluid control systems can then be operated to flow at least a portion of the blood to the glucose measurement system (9), where the glucose concentration of the blood (or other analyte property, if another analyte sensor is employed) can be determined. The fluid control systems can then be operated to flow the blood, including at least a portion of the blood measured by the glucose measurement system, into either the left or right side of the system and then back to the patient. As desired, the fluid control systems can be operated to flow maintenance fluid from the maintenance fluid source (18) through the glucose measurement system (9) to the waste channel (4) to facilitate cleaning or calibration of the system. The fluid control systems can also be operated to flow maintenance fluid through the left and right sides to facilitate cleaning of the tubing or other fluid transport mechanisms. The fluid control systems can also be operated to flow maintenance fluid into the patient for example at a tow rate to maintain open access to the circulatory system of the patient.

Push Pull with Additional Path.

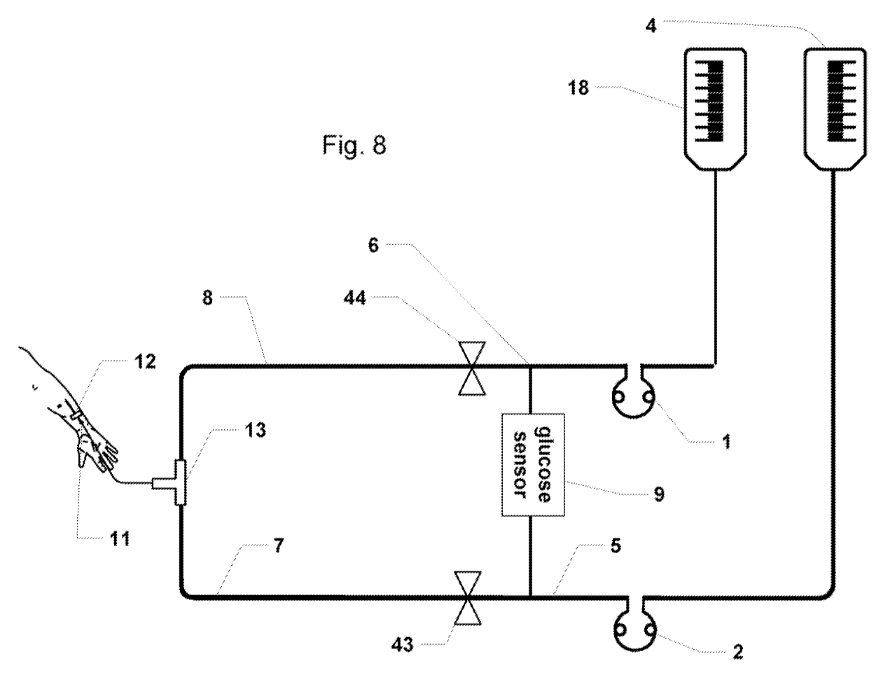

FIG. 8 is a schematic illustration of an example embodiment. The system comprises a catheter (or similar blood access device) (12) in fluid communication with the vascular system of a patient. A tubing extension (11) (if required) extends from the catheter (12) to a junction (13) A first side of the junction (13) connects with fluid transport apparatus (8) such as tubing (for reference purposes called the “left side” of the blood loop); a second side of the junction (13) connects with fluid transport apparatus (7) such as tubing (for reference purposes called the “right side” of the blood loop). A pinch valve (44) controls flow between the left side (8) of the blood loop and an intermediate fluid section (6). A pump (1) mounts between the intermediate fluid section (6) and a source of saline such as a bag (18). A pinch valve (43) controls flow between the right side (7) of the blood loop and an intermediate fluid section (5). A pump (2) mounts between the intermediate fluid section (5) and a waste channel such as a bag (4). A glucose sensor (9) mounts between the two intermediate fluid sections (6, 5). Elements and their operation are further described below.

Blood Sample and Measurement Process.
1. Blood is removed from the patient via the blood pump (1) while pinch valve (44) is open and pinch valve (43) is closed.
2. At the end of the draw blood is diverted into the tubing path containing the measurement cell (9) by activation of pump (2) with the concurrent closure of pinch valve (43).
3. A volume of blood appropriate for the measurement can be pulled into (or past as needed) glucose sensor (9) and into tubing (5). The rate at which the blood is pulled into tubing (5) can be performed such that the draw time is minimized.
4. At this juncture the re-infusion process can be initiated. Pump (2) initiates a re-infusion of the blood at a rate consistent with the measurement of the blood sample. In general terms this rate is stow as the blood simply needs to flow at a rate that results in a substantially constant sensor sampling. Concurrently, pump (1) initiates a re-infusion of the blood.
5. As has been described previously, the amount of saline infused into the patient can be controlled via the use of the flush line (7).
6. The system can then be completely cleaned via the use of the two pumps (1, 2) as well as pinch valves (43 44).

Characteristics of Push Pull with Additional Path. This example embodiment can perform measurement and infusion concurrently. In the previously-described push-pull system the withdrawal, measurement, and re-infusion generally occur in a sequential manner. In the system of FIG. 8 the measurement process can be done in parallel with the infusion. The reduction in overall cycle time can be approximately 30%.

In addition to the reduction in total cycle time, the system has the ability to provide independent cleaning paths. By closing or opening the pinch valves in combination with the two pumps, the system can create bi-directional flows and clean the sensor measurement cell independent of the rest of the circuit. Such independent cleaning paths are especially useful when managing either complete or partial occlusions.

The push pull with additional path system as illustrated in FIG. 8 is an example embodiment of one possible configuration. The pump mechanism can be moved to the portion of tubing between the junction leading to the glucose sensor and the patient. Many other pump and flow control devices can be used to create the operational objectives defined above. Additionally, the system can be realized with only one pump.

The push pull with additional path system as illustrated in FIG. 8 also has the advantage of being able to deliver a sample to the glucose sensor without it being preceded by saline. As the blood is withdrawn up the left side of the circuit the saline/blood transition area can be moved beyond the location where blood sensor (9) connects with tubing (6). At this point the blood that is moved into sensor (9) could have a very small or no leading saline boundary. The lack of such a leading saline boundary can facilitate the use of the system with existing blood glucose meters. Typically, these meters make the assumption that all fluid in contact with the disposal strip is blood, not a mixture of blood and saline.

Sample Isolation at the Arm with Subsequent Discard.

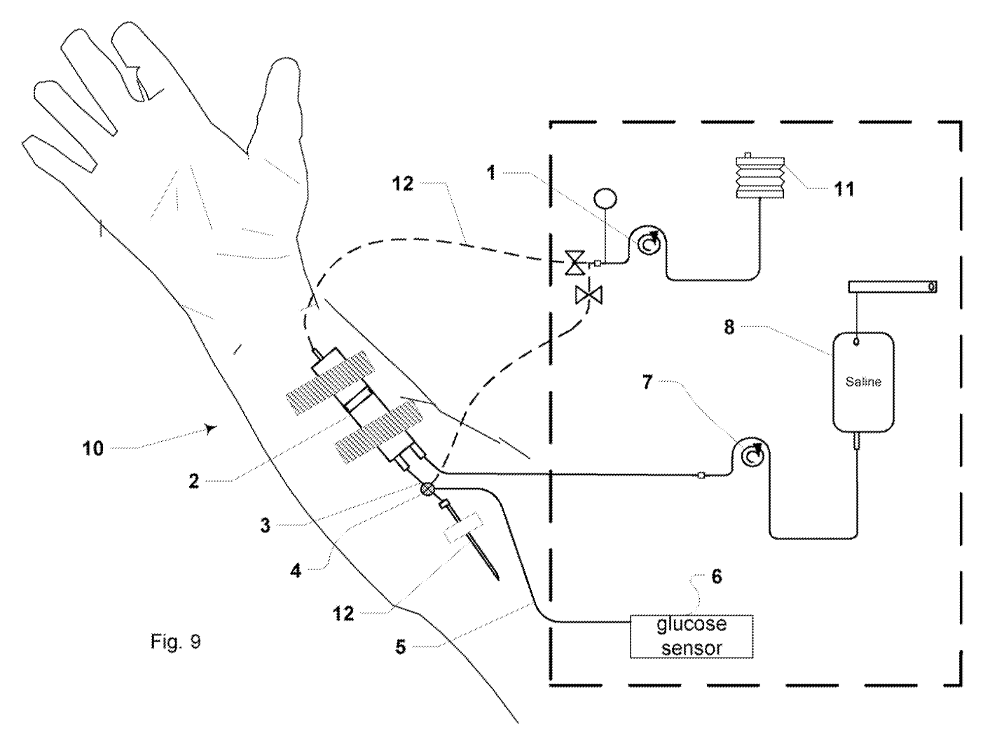

FIG. 9 is a schematic illustration of an example embodiment that allows a blood sample for measurement to be isolated at a point near the patient and then transported to the instrument for measurement. The system shown does not require electronic systems attached to the patient. A hydraulically actuated syringe (10) is provided, with a pump (1) and saline reservoir (11) and tubing (12) provided to control actuation of the syringe (10). A catheter (12) is in fluid communication with the vascular system of a patient. The syringe (10) can mount such that it draws blood from the patient via the catheter (12). A valve (4) controls flow between the catheter and a transport mechanism (5) in fluid communication with a glucose measurement device (6). The syringe (10) is also in fluid communication with a pump (7) and an associated fluid reservoir such as a bag of saline (8). The system can be described as one that is remotely activated by hydraulic action. Elements of the system and their operation are further described below.

Blood Sample and Measurement Process.
1. The blood is withdrawn from the patient using hydraulically activated syringe (1). The syringe is controlled by pump (1).
2. The removal of some blood into syringe (2) creates an undiluted and clean blood sample in catheter (3).
3. Valve (4) is activated into an open position such that a small sample of blood is diverted into tubing pathway (5). The blood is subsequently transported to measurement cell (6) for measurement. The blood transport into glucose sensor (6) can be via air, saline or other appropriate substances.
4. The blood in syringe (2) is re-infused by activation of pump (1). Following re-infusion of the blood the system can be cleaned with saline by activation of pump (7),
5. The blood located in the measurement cell is measured and subsequently discarded to waste (not shown).

The system can be operated in several different modes. The delivery of a small sample to the measurement site can be easily accomplished by the use of air gaps to isolate the sample from other fluids that can otherwise tend to dilute the sample. In this measurement method the volume of the sample does not need to be tightly controlled and the measurement system measures the glucose (mg/dl) in the sensor cell,

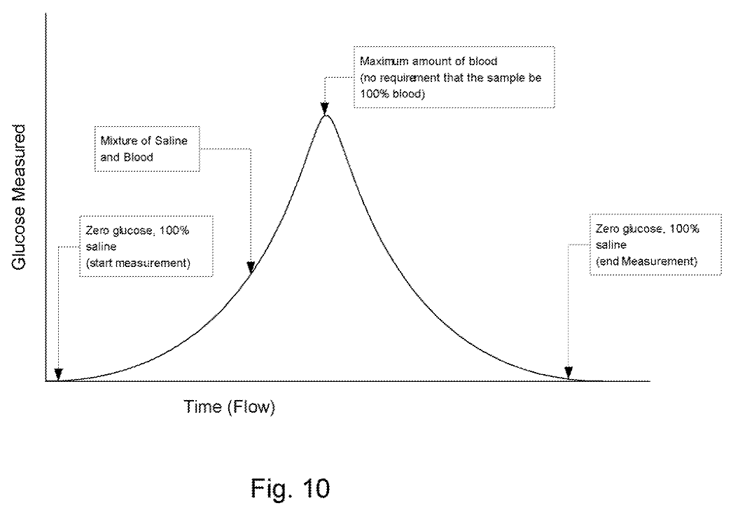

An alternative approach involves either reproducible control of the volume of blood or determination of the volume of blood and integration of the total amount of glucose measured, as illustrated in FIG. 10. The blood sample can then undergo significant mixing with the transport fluids since there is no requirement that an undiluted sample be delivered to the sensor cell. The system can effectively determine the total amount of glucose measured. The total amount of glucose could be determined by a simple integration for the area under the curve with both the total amount of glucose known and the volume of blood processed, an accurate determination of the blood glucose can be made.

Characteristics of Sample Isolation at the Arm with Subsequent Discard. The total amount of blood removed during the sampling process is minimized by this system. Additionally the amount of saline infused is also minimized.

The pressure needed to withdrawal the blood sample can be monitored for partial or complete occlusion. If such a situation is observed the flush pump can be used to either clean the catheter or to clean the circuit over to the measurement cell. In addition the activation of the flush pump in conjunction with the hydraulic syringe can be used to create rapid flows, turbulent flows and to isolate particular components of the circuit for cleaning.

Sample Isolation System.

FIG. 15 is a schematic illustration of a blood access system according to the present invention. The system comprises a catheter (or similar blood access device) (12) in fluid communication with the vascular system of a patient. A tubing extension (51) (if required) extends from the catheter (12) to a junction (13). A first side of the junction (13) connects with fluid transport apparatus (52) such as tubing; a second side of the junction (13) connects with fluid transport apparatus (53) such as tubing. A sample system (38) is in fluid communication with fluid transport apparatus (52). A one-way fluid control device (32) (e.g., a check valve) receives connects so as to receive fluid from fluid transport apparatus (53) and deliver to a junction (33). A first side of the junction (33) is in fluid communication with a drive system (39); a second side of the junction is in fluid communication with fluid transport apparatus (54) such as tubing. A sensor (49) is connected so as to receive fluid from fluid transport apparatus (54). A waste container or channel (45) is connected so as to receive fluid from the sensor (49). (53), (32) and (33) can be separate components or be integrated as a single component to minimize dead space volume between the functions of each component.

In operation, the sample system (38) draws blood from the patient into fluid transport apparatus (51) and (52). After a sufficient volume of blood has been drawn into (51) and (A2), the sample system (38) pushed blood from (52)through one-way device (32) to junction (33). Drive system (39) pushes a “plug” into junction (33), where a plug can comprise a quantity of a substance relatively immiscible with blood and suitable for transport through tubing or other components in transport apparatus (54) and suitable for transport through sensor (49) without contamination of the sensor (49). Examples of suitable plug materials include air, inert gases, polyethylene glycol (PEG), or other similar materials. An alternative type of plug can comprise fixing or clotting the blood at the leading and trailing edges. Specifically, glutaraldehyde is a substance that causes the hemoglobin in the red blood cell to become gelatinous. The net result is a gelatinous plug that can be used effectively to separate the blood used for measurement from the surrounding fluid. After the initial plug is pushed into junction (33), sample system (38) pushes additional fluid into (52), forcing blood from (53) past junction (33) forcing the initial plug in front of the blood into transport apparatus (54). Sample system can push blood into (52), or can push another suitable fluid such as saline into (52), or can reduce the volume of (52), or any other method that moves the blood in (8) into junction ($33) and transport apparatus (54). Once a sufficient quantity of blood is present in transport apparatus (54), drive system (39) can push a second or trailing plug into junction (33). Transport system (39) can then push the plug-blood-plug packet through transport apparatus (54) so that the blood can be measured by sensor (49). The blood can be immediately pushed to waste (45), or pushed to waste by the transport of a subsequent sample. Since the blood in transport apparatus (54) is surrounded by relatively immiscible plugs., and since the drive system (39) can push the plug-blood-plug packet using techniques optimized for transport (egg., pressurized air or other gas, or mechanical compression of transport apparatus (54)), the blood can be transported more quickly, and over greater distances, than if the patient's blood or saline were used as the motive medium.

Sample Isolation Though Use of Air Gaps.

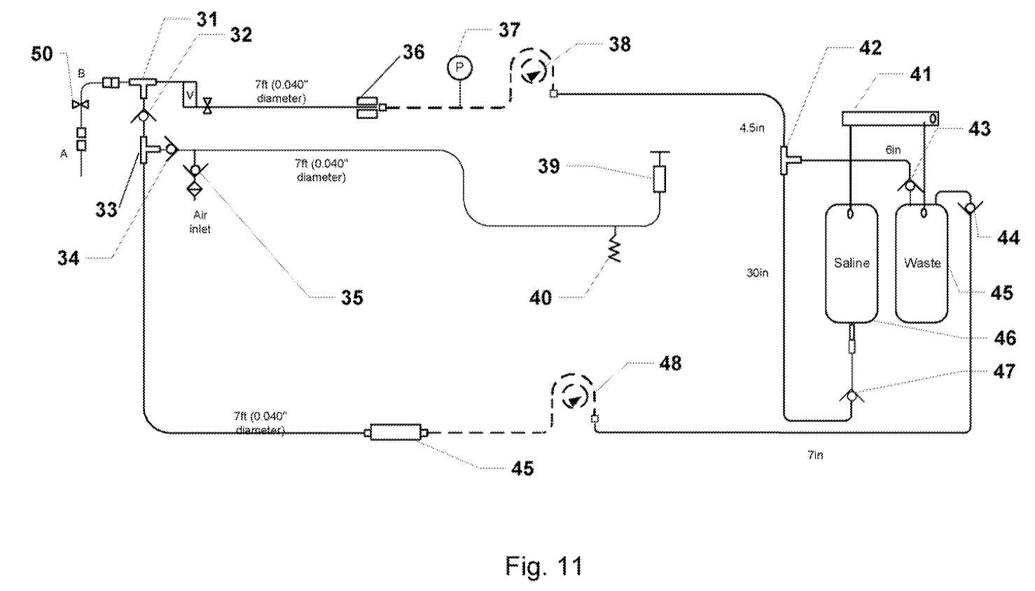

FIG. 11 is a schematic illustration of an example embodiment that allows a blood sample for measurement to be isolated at a point near the patient and then transported to the instrument for measurement through the use of leading and the following air gaps. The system is able to effectively introduce air gaps through a series of one-way valves while concurrently preventing air from being infused into the patient. The system is adapted to connect with the circulation system of a patient through blood access device (50). A recirculating junction (31) has a first port in fluid communication with a patient, with a second port in fluid communication with a one-way (or check) valve (32). The valve (32) allows flow only away from the recirculating junction (31) toward a port of a second junction (33). A second port of the second junction (33) is in fluid communication with a one-way valve (34), which allows flow only towards the second junction (33). The one-way valve (34) is in fluid communication with another one-way valve (35) and with an air pump (39). The communication between the air pump (39) and the one-way valve (35) can be protected with a pressure relief valve (40). The one-way valve (35) accepts air from an external source. A third port of the second junction (33) is in fluid communication with a glucose sensor (49), which in turn is in fluid communication with a pump (48), and then to a one-way valve (44) that allows flow from the pump to a waste channel such as a waste bag (45). Another port of recirculating junction (31) is in fluid communication with a pump (38). The path from the recirculating junction (31) to the pump (38) can also interface with a pressure sensor (37) and an air detector (36). The pump (38) is in fluid communication with a junction (42). Another port of junction (42) is in fluid communication with a one-way valve (43) that allows fluid flow from the pump (38) to a waste channel such as waste bag (45). Another port of junction (42) is in fluid communication with a one-way valve (47) that allows fluid flow from a saline source such as saline bag (46) to the pump (38). Manual pinch clamps and access ports can be provided at various locations to allow disconnection and access, e.g., to allow disconnection from the patient.

Blood Sample and Measurement Process.
1. Blood is withdrawn from the patient utilizing the blood pump until a clean or uncontaminated sample has been pulled pass the recirculation junction.
2. Additional brood is withdrawn from the patient by activation of the pump labeled recirculation pump. Blood is pulled to the air junction.
3. An air plug is created by pulling back on the air pump (39). The one-way valve at the air intake allows air into the tubing set for the formation of a small air gap.
4. The air gap is infused through valve (34) to create a leading air gap in junction(33) which is located at the leading edge of the uncontaminated blood sample.
5. The recirculation pump (48) then withdraws blood from the patient until an appropriate volume of uncontaminated blood has been procured.
6. The air pump (39) is again operated in the mode to create a second air gap that will be used as a trailing air segment.
7. The second air plug is infused through valve (34) to create a following air gap.
8. The blood residing in the line leading to the blood pump is infused into the patient.
9. The blood sample with leading and trailing air gaps is now transported over to the glucose sensor (45). Once in contact with the glucose sensor, an accurate glucose measurement can be made.
10. Following completion of the measurement sample is discarded to waste (45).
11. The circuit is now completely filled with saline and additional cleaning the circuit can be performed.

Characteristics of sample isolation by leading and trailing air gaps. There are a number of advantages associated with this isolation system, specifically the total amount of blood removed from the patient can be significantly less due to the fact that the blood sample is isolated at a point very close to the patient. The isolation of the blood sample and transportation of that small amount of blood to the measurement has advantages relative to a system that transports a large amount of blood to the measurement site. The fact that a small amount of total blood is withdrawn results in decreased overall measurement time or dwell time. The decreased amount of blood removed enables the system to operate at lower overall withdrawal rates and with lower pressures. Additionally, the isolation the blood sample has the advantage at the isolated sample can be measured for a prolonged period of time, can be altered in ways that are incompatible with re-infusion into the patient. Due to pressure monitoring on the blood withdrawal and the possible inclusion of a second pressure sensor on the recirculation side of the circuit (not shown), the circuit design has extremely good occlusion management capabilities. The isolation of the blood sample and inability to re-infuse the sample due to the use of one-way valves, can create the opportunity to use non-sterile measurement methodologies.

Hematocrit Influence on Withdrawal Pressures.

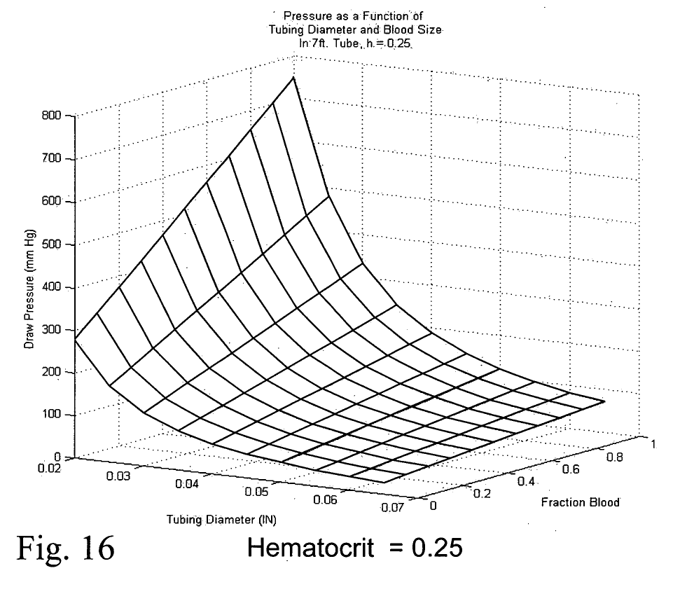

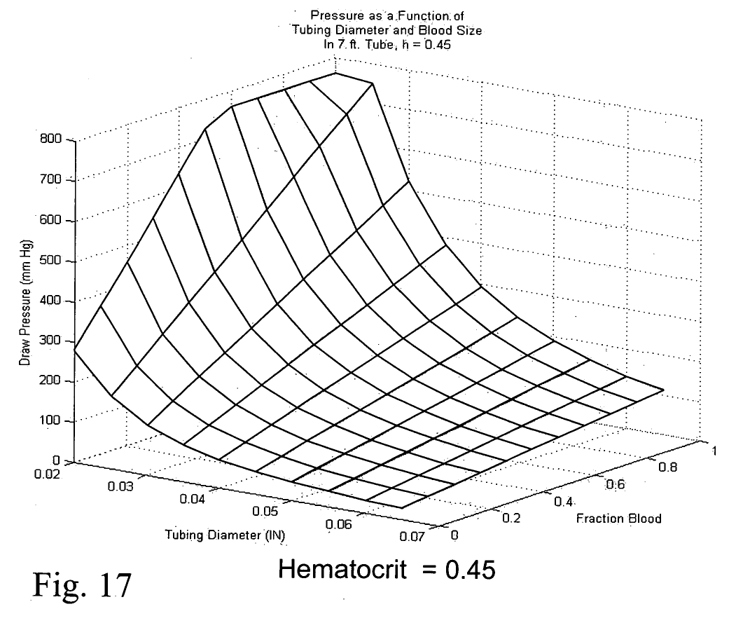

FIG. 16 is an illustration of a relationship between withdrawal pressure, tubing diameter and blood fraction at a fixed hematocrit. As used here blood fraction is the percent volume occupied by blood assuming a 7 foot length of tubing. FIG. 16 depicts this relationship assuming a hematocrit of 25%. FIG. 17 is the same information but assuming a hematocrit of 45%. Examination of these graphs shows significant pressure increases associated with increasing hematocrit, decreasing tube size and increasing blood fraction. In general terms, it can be desirable to use smaller tubing as the amount of blood required is less and the length of the blood saline junction is less. These generally desirable attributes are offset by the fact that smaller tubing requires higher pump pressures Comparison of FIG. 16 with FIG. 17 also shows that there is strong sensitivity to the fraction of blood and the tubing diameter. With a glucose measurement methodology that requires only a small sample of blood, it can be desirable to use a smaller blood fraction which results in lower overall circuit pressures.

Hematocrit Influence on Blood Saline Junction.

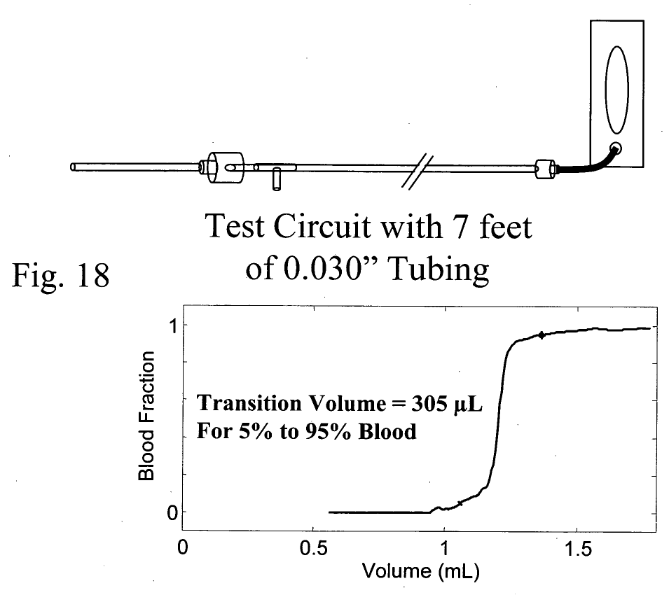

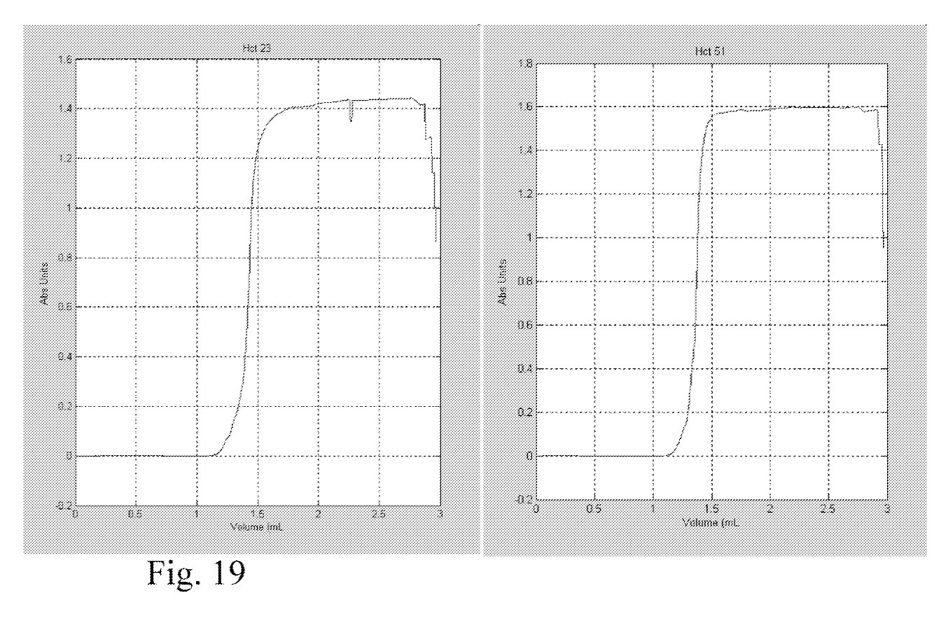

FIG. 18 shows a test system used to determine the amount of blood saline mixing that occurs during transport of the blood through the tubing, including the luer fittings, junctions, and the subsequent filling of the optical cuvette. In testing., the system is initially fitted with saline and blood is withdrawn into the tubing set. An optical measurement is performed throughout the withdrawal cycle. As the transition from saline to blood occurs the optical density indicated by the optical measurement of the sample changes. A transition volume representing the volume needed to progress from 5% absorbance to 95% absorbance can be calculated from the recorded data. FIG. 19 shows the results from the above test apparatus for two hematocrit levels, 23% and 51%. As can be seen from FIG. 19, the transition volume is greater for the lower hematocrit blood. The dependence of the transition volume on hematocrit level can be used as an operating parameter for improved blood circuit operation.

Use of Blood/Saline Transition for Measurement Predictions

As shown in FIG. 19, the transition from saline to blood is a systematic and a repeatable transition. By using the fact that the transition is repeatable for a given hematocrit, the measurement process can be initiated at the start of this transition zone. In the case of 23% hematocrit, the measurement process could be initiated falling withdrawal of 1.5 ml. The measurement process could then account for the fact that there is a known dilution profile as a function of withdrawal amount. For, example the system can make measurements at discrete intervals and project to the correct undiluted glucose concentration.

Modified Operation of Push Pull System with Two Peristaltic Pumps.

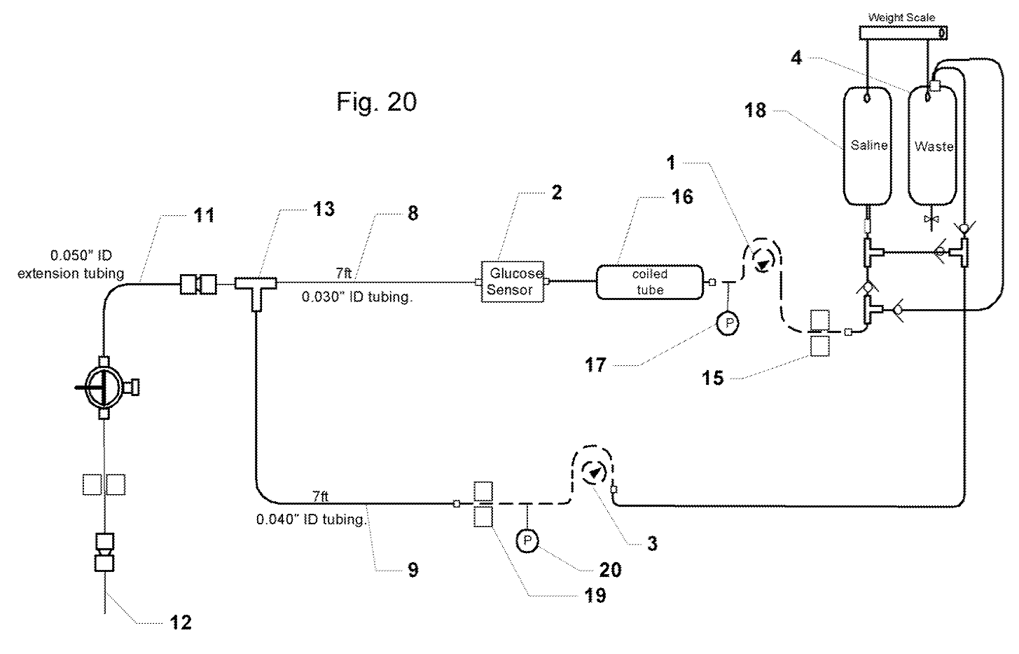

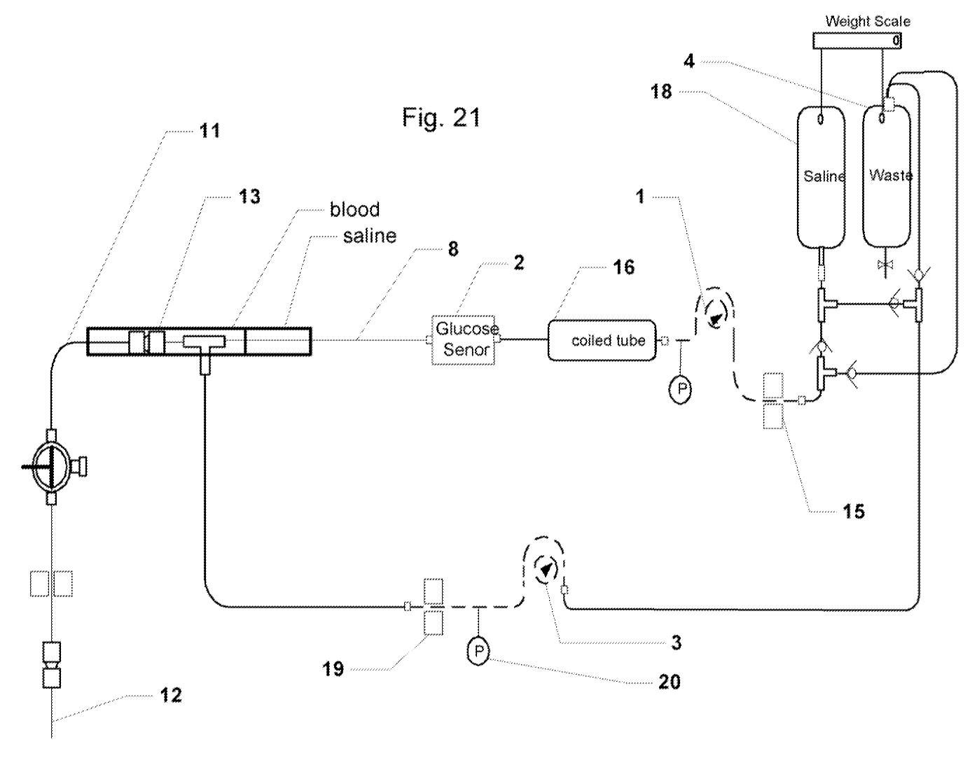

FIG. 20 is a schematic illustration of a blood access system based upon a push-pull mechanism with a second circuit provided to prevent fluid overload in the patient. The circuit is similar to that depicted in FIG. 5 but is operated in manner that optimizes several operational parameters. The system comprises a catheter (or similar blood access device) (12) in fluid communication with the vascular system of a patient. A tubing extension (11) (if required) extends from the catheter (12) to a junction (13). A first side of the junction (13) connects with fluid transport apparatus (8) such as tubing (for reference purposes called the “left side” of the blood loop) a second side of the junction (13) connects with fluid transport apparatus (9) such as tubing (for reference purposes called the ““right side” of the blood loop). An air detector (15) that can serve as a leak detector, a pressure measurement device (17), and a glucose sensor (2) mounted on the left side of the blood loop. A tubing reservoir (16) mounts with the left side of the blood loop, and is in fluid communication with a blood pump (1). Blood pump (1) is in fluid communication with a reservoir (18) of fluid such as saline. A second air detector (19) that can serve as a leak detector mounts with the right side of the blood loop. A second blood pump (3) mounts with the right side of the blood loop, and is in fluid communication with a receptacle or channel for waste, depicted in the figure as a bag (4). A second pressure sensor (20) can mount with the right side of the blood loop. An additional element shown in FIG. 20 is the specific identification of an extension set. The extension set is a small length of tubing used between the standard catheter and the blood access circuit. This extension set adds additional dead volume and other junctions that can be problematic from cleaning perspective. Elements of the system and their operation are further described below.

Modified operations. As shown in the preceding plots, high hematocrit blood requires a large pressure gradient but the increased viscosity of the blood results in smaller transition volumes. Lower hematocrit blood is the opposite, requiring lower pressures and larger transition volumes. In simple terms, the device can be operated to withdraw only enough blood such that an undiluted sample can be tested by the glucose sensor. Due to the lower transition volumes associated with higher hematocrit blood the amount of blood drawn can be appreciably smaller than the volume needed with lower hematocrit blood. For operation on a human subject the following general criteria can be desirable,
1) Minimize the total amount of blood withdrawn, this lowers overall exposure of blood to non-human surfaces.
2) Minimize the maximum pressure needed for withdrawal this reduces the power requirements and pump sizes needed to move the blood,
3) Utilize the smallest tubing diameter possible, this reduces the blood volume and reduces mixing at the blood/saline interface.
4) Clean out the tubing between the blood vessel and the junction as soon as possible, this can help reduce the likelihood of clotting at this location.

Blood Sample and Measurement Process—Subsequent Blood Pump.

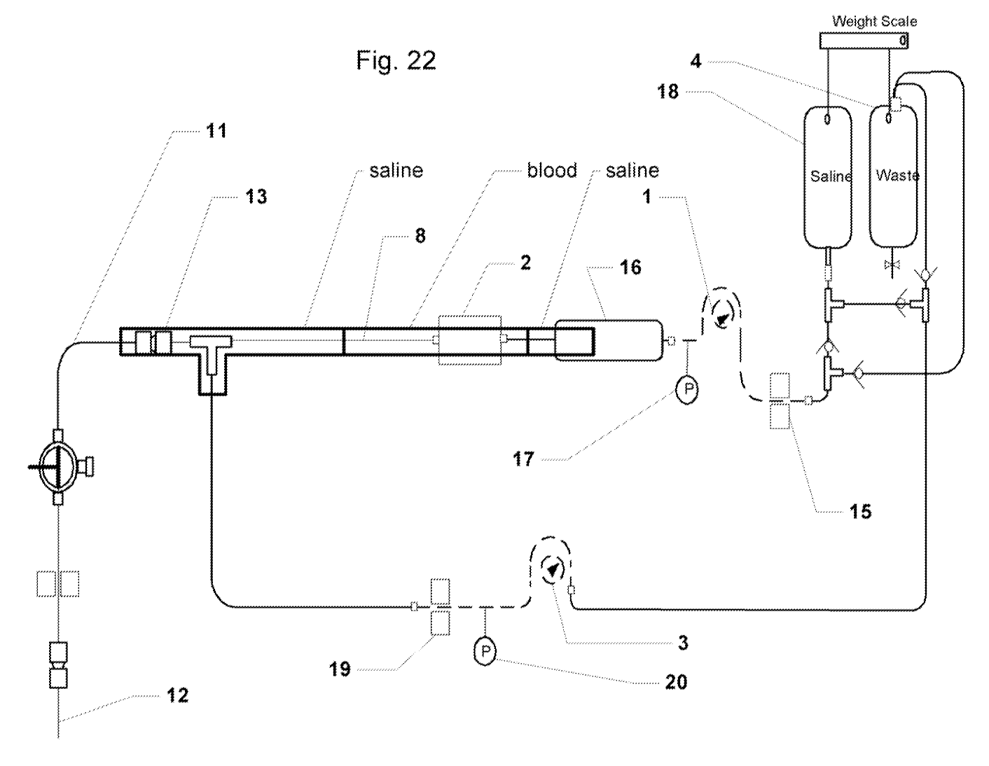

The example circuit shown in FIG. 20 can be operated in the manner that balances the four potentially competing objectives set forth above. The system can achieve improved performance by taking advantage of the small amount of undiluted blood sample actually required for sensor operation. Notice that, while a blood sample must be transported through the left side, the left side does not need to be completely filled with blood. Saline (or another suitable fluid or material) can be used to push a blood sample to the sensor. An example sequence of steps are set forth below:
1. Pump (1) initiates a blood draw by drawing blood through junction(13).
2. The withdrawal continues until enough blood has been withdrawn past the junction of junction (13) and the right side (9) of the loop such than an undiluted and appropriately sized blood segment can be delivered to the glucose sensor, as illustrated schematically in FIG. 21. As mentioned above the amount of blood needed can be hematocrit dependent. Therefore, the amount of blood withdrawn past the junction (13) can be controlled based on measured hematocrit: smaller blood segments with higher hematocrit and larger blood segments with lower hematocrit. Following the withdrawal of an appropriate blood segment, the blood pump (1) continues to operate but the flush pump (3) is also turned on, as illustrated schematically in FIG. 22. The flush pump (3) can be operated at a rate equivalent to or greater than the blood pump (1). If operated at a rate greater then the blood pump (1), the flow rate imbalance forces saline (or other suitable fluid or material) into the right side (8$) transporting the blood sample segment to the sensor, and also back into the extension tubing (11), cleaning the junction (13) and the extension tubing (11). As an example, the flush pump can initially be actuated at very high rate to rapidly clean the tubing connected to the patient and then decreased to primarily facilitate transport of the blood segment to the sensor measurement site.
3. As blood passes through the sensor measurement cell (2), it is stored in the tubing reservoir (16).
4. Sensor measurements can be made during this withdrawal period.
5. The blood can be moved back and forth over the sensor for an increased measurement performance (in some sensor embodiments) without the requirement for greater blood volumes.
4. Following completion of the blood measurement, the blood can be re-infused into the patient by reversing the direction of pump (1).
5. Sensor measurements can also be made during the re-infusion period.
6. As the mixed blood-saline passes through the junction(13), it becomes progressively more dilute.
7. Following re-infusion of the majority of the blood, flush pump (3) is turned on at a rate equal to or less than the rate of pump (1). If less than the rate of pump (1) then there is a small amount of saline re-infused into the patient. If operated at the same rate then there is substantially no net infusion into the patient. A small amount of residual blood mixed with the saline is taken to the waste bag (4).
8. This process results in a washing of the system with saline.
9. Additional system cleaning is possible through an agitation mode. In this mode the fluid is moved forward and back such that turbulence in the flow occurs. During this process both pumps can be used.
10. As a final step, the tubing between the junction and the patient, including the extension set (11), can be further cleaned by the infusion of saline by both the flush pump and the blood pump. The use of both pumps in combination increases the overall for flow through this tubing area and helps to create turbulent flow that aids in cleaning
10. Between blood samplings, the system can be placed in a keep vein open mode (KVO). In this mode a small amount of saline can be infused to keep the blood access point open.

Characteristics of Modified Push Pull Example Embodiment. The example embodiment of FIG. 20 has similar characteristics as those of the example embodiment depicted in FIG. 5, and has the additional advantage of using a smaller overall blood withdrawal amount. The example embodiment of FIG. 20 can also rapidly clean the tubing section between the junction and the patient, and operate with reduced overall pressures. Additionally, the circuit can be operated in a manner where the hematocrit of the patient's blood is used to optimize circuit performance by modifying the pump control. The use of hematocrit as a control variable can further reduce the amount of blood withdrawn and the maximum pressures required.

The use of the flush line in a bidirectional mode has several distinct advantages. During the final washing the rate of flow to the extension set at reasonable pressures can be greater than those obtained by using only the blood pump. In addition to improved washing, the flush line can be used to “park” a diluted leading segment. Specifically, the initial draw can be performed by the flush pump (3) such that the blood saline junction is moved into the right side of the circuit. After the blood/saline junction has passed and an undiluted sample has progressed to the T-junction, the left side of the circuit can be activated via the blood pump and a blood segment with a better defined saline/blood boundary transported to the measurement sensor. As leuer fittings between the extension set and the standard catheter are a major source of blood/saline mixing the ability to “park” this mixed segment can be advantageous.

Central Venous Operation. The ability to “park” the blood segment can be especially important when using the system on a central venous catheter (CVC). All figures in this disclosure show the use of the system on peripheral venous catheters, which typically have volumes of less than 500 μL. In the case of a central venous catheter, the volumes in the catheter can become quite large, around 1 ml, since that they can extend for up to 3 feet in the patient. This increased volume and length of tubing increases the amount of dead volume that must be withdrawn and increases the mixing at with the blood/saline boundary. Given the larger volumes preceding the undiluted blood segment, it can be desirable to “park” the blood from the CVC near the access location instead of transporting it through 7 feet of tubing to the measurement sensor. In operation, it has been found advantageous to use larger diameter tubing in the right side of the circuit and smaller diameter tubing in the left side. The use of larger diameter tubing enables a more rapid draw from the CVC line, white smaller tubing used to connect the glucose sensor has been found to minimize the total volume of blood removed from the patient.

Push Pull System with Two Peristaltic Pumps and Modified Sensor Location.

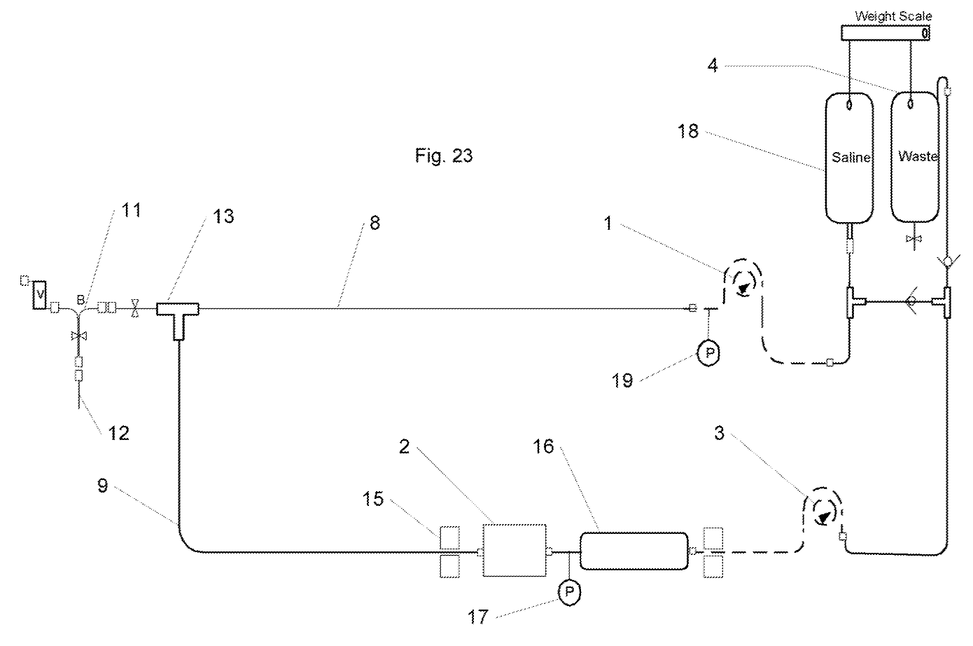

FIG. 23 is a schematic illustration of an example blood access system implemented based upon a pull-push mechanism. The example circuit is similar to that depicted in FIG. 20 but the glucose sensor is in a different location. The system comprises a catheter (or similar blood access device) (12) in fluid communication with the vascular system of a patient. A tubing extension (11) (if required) extends from the catheter (12) to a junction (13). A first side of the junction (13) connects with fluid transport apparatus (8) such as tubing (for reference purposes called the “left side” of the blood loop); a second side of the junction (13) connects with fluid transport apparatus (9) such as tubing (for reference purposes called the “right side” of the blood loop). An air detector (15) that can serve as a leak detector, a pressure measurement device (17), and a glucose sensor (2) mount on the right side of the blood loop. A tubing reservoir 16 mounts with the right side of the blood loop, and is in fluid communication with a blood pump (3), which is in fluid communication with a receptacle or channel for waste, depicted in the figure as a bag (4). A blood pump (1) mounts with the left side (8) of the system, and is in fluid communication with a reservoir (18) of fluid such as saline. A blood detector (19) serves as a leak detector mounts on the left side of the blood loop. An extension tubing set (11) can (and in many applications, will be required to) mount between the blood access device (12) and the junction (13). An extension set is generally a small length of tubing used to between a standard catheter and the blood access circuit. This extension set adds additional dead volume to the system, and adds other junctions that can be complicate cleaning. Elements of the system and their operation are further described below.

Blood sample and measurement process—Subsequent Blood Sampling. In operation the circuit shown in FIG. 23 operates in a manner very similar to the “park” method described above. A blood sample can be drawn into the right side (9) and transported to the glucose measurement site, or a portion of the blood can be drawn and parked into the left side (8) first (as discussed more fully above). The following example operational sequence can be suitable, other sequences can also be used. For an initial sample, the tubing between the patient and the pump (1) can be filled with saline as a start condition. Subsequent measurements can be achieved with operation as follows:
1. Pump (1) initiates the blood draw by drawing blood up through junction(13).
2. The withdrawal continues as blood passes through the junction (13) until an undiluted segment of blood is present at the junction (13)
3. Pump (1) stops and pump (3) draws the undiluted segment toward the glucose sensor (2).
4. Following removal of an appropriate blood segment, pump (1) can be activated in a manner that cleans the tubing from the junction (1 3) to the patient and concurrently helps to push the undiluted segment to the glucose sensor (2).
5. Following completion of the glucose measurement, pump (3) can be activated such that majority of blood is re-infused into the patient.
6. At the point the majority of blood has been returned to the patient, pump (1) can be activated and the direction of pump (3) reversed such that the circuit is effectively cleaned. The small amount of residual blood mixed with the saline is taken to the waste bag (4).
7. Between blood samplings, the system can be placed in a keep vein open mode (KVO).

In this mode a small amount of saline can be infused to keep the blood access point open.

Advantages of Pressure measurement. The systems as shown throughout this disclosure can use two pressure measurement devices which may or may not be specifically identified in each figure. These devices can be utilized to identify occlusions in the circuit during withdrawal and infusion as well as the location of the occlusion. Additionally, the pressure sensors can be used to effectively estimate the hematocrit of the blood. The pressure transducer on the flush line effectively measures pressures close to the patient, while the pressure measurement device on the blood access line measures the pressure at the blood pump. The pressure gradient is a function of volume and hematocrit. The volume pumped is known, and thus the pressure gradient can be used to estimate the hematocrit of the blood being withdrawn.

FIG. 20 shows the use of two peristaltic pumps. In use peristaltic pumps create a pressure wave when the tubing is no longer compressed by the roller mechanism. The characteristics of this pressure wave when transmitted through blood or saline are defined. When the air or an air bubble is present in the system the overall compliance of the system is dramatically altered and the characteristics of this pressure wave are altered. By using one or both of the pressure measurement devices as a pressure wave characterization system, the device can detect the presence of air emboli in the circuit.

The particular sizes and equipment discussed above are cited merely to illustrate particular embodiments of the invention. It is contemplated that the use of the invention can involve components having different sizes and characteristics. It is intended that the scope of the invention be defined by the claims appended hereto.

What is claimed is:

1. An apparatus for measuring an analyte in blood taken from a patient, comprising;
a. An analyte measurement system;
b. A fluidics system, adapted to remove blood from a body, transport a portion of the removed blood to the analyte measurement system for measurement, infuse a portion of the blood measured by the analyte measurement system back into the patient, flow a maintenance substance to the analyte measurement system without infusing a substantial amount of the maintenance substance into the patient, and flow at least a portion of the maintenance substance from the analyte measurement system to a waste channel.

2. An apparatus as in claim 1, wherein the maintenance substance is a fluid that cleans the analyte measurement system.

3. An apparatus as in claim 1, wherein the maintenance substance is a fluid that provides a calibration measurement using the analyte measurement system.

4. An apparatus as in claim 1, wherein the analyte is glucose, and the analyte measurement device is a glucose measurement device.

5. An apparatus as in claim 4, wherein the glucose measurement device comprises one or more of; electrochemical sensor, microfluidic sensor, micropost sensor, fluorescent measurement device, and an enzyme-based sensor, a spectroscopic measurement sensor.

6. An apparatus for determining an analyte property in blood, comprising:
a. a blood removal element, adapted to communicate blood with the circulatory system of a patient;
b. a fluid junction having three ports in fluid communication with each other, the first port in fluid communication with the blood removal element;
c. a source of maintenance fluid;
d. a channel for waste;
e. an analyte sensor having first and second fluid ports;
f. a first fluid control system, in fluid communication with and adapted to control fluid flow between the second port of the junction, the first port of the analyte sensor, and the source of maintenance fluids;
g. a second fluid control system, in fluid communication with and adapted to control fluid flow between the third port of the junction, the second port of the analyte sensor, and the waste channel.

7. An apparatus as in claim 6, wherein the first fluid control system comprises:
a. a first pump, connected between the second port of the junction and the first port of the analyte sensor;
b. a first flow control element, connected between the first port of the analyte sensor and the source of maintenance fluid.

8. An apparatus as in claim 6, wherein the first fluid control system comprises:
a. a first flow control element, connected between the second port of the junction and the first port of the analyte sensor;
b. a first pump, connected between the first port of the analyte sensor and the source of maintenance fluid.

9. An apparatus as in claim 6, wherein the first fluid control system comprises:
a. a first pump, connected between the third port of the junction and the second port of the analyte sensor;
b. a first flow control element, connected between the second port of the analyte sensor and the waste channel.

10. An apparatus as in claim 6, wherein the first fluid control system comprises:
a. a first flow control element, connected between the third port of the junction and the second port of the analyte sensor;
b. a first pump, connected between the second port of the analyte sensor and the waste channel.

11. An apparatus as in claim 8, wherein the second fluid control system comprises:
a. a second flow control element, connected between the third port of the junction and the second port of the analyte sensor;
b. a second pump, connected between the second port of the analyte sensor and the waste channel.

12. An apparatus as in claim 8, wherein the second fluid control system comprises:
a. a second pump, connected between the third port of the junction and the second port of the analyte sensor;
b. a second flow control element, connected between the second port of the analyte sensor and the waste channel.

13. An apparatus as in claim 7, wherein the second fluid control system comprises:
a. a second flow control element, connected between the third port of the junction and the second port of the analyte sensor;
b. a second pump, connected between the second port of the analyte sensor and the waste channel.

14. An apparatus as in claim 6, wherein the analyte sensor is a glucose sensor.

15. An apparatus as in claim 6, wherein the waste channel comprises a bag adapted to receive and store waste fluid.

16. An apparatus as in claim 6, wherein the maintenance fluid source comprises a bag containing saline solution.

17. A method of determining an analyte property of blood using an apparatus as in claim 6, comprising:
a. operating the first fluid control system and the second fluid control system to transport blood from the blood removal element to either the first or second fluid control system;
b. operating the first fluid control system and the second fluid control system to transport at least a portion of the blood transported in step a to the analyte sensor;
c. determining the analyte property using the analyte sensor.

18. A method as in claim 17, further comprising d) operating the first fluid control system and the second fluid control system to transport at least a portion of the blood in the analyte sensor to the blood removal element.

19. A method as in claim 17, further comprising d) operating the first fluid control system and the second fluid control system to transport maintenance fluid from the source of maintenance fluid through the analyte sensor to the waste channel, without transporting a substantial volume of maintenance fluid to the circulatory system of the patient.

20. A method as in claim 19, wherein the first fluid control system and the second fluid control system are operated such that variable fluid flow is attained during step d.

21. A method as in claim 19, wherein the first fluid control system and the second fluid control system are operated such that fluid flows through the analyte sensor in opposite directions during two distinct times in step d.

22. A method as in claim 17, wherein step b comprises operating the first fluid control system and the second fluid control system such that there is substantially no fluid flow through the blood removal element during step b.

23. An apparatus as in claim 6, wherein the maintenance fluid produces a predetermined response from the analyte sensor.

24. A method as in claim 17, wherein the maintenance fluid comprises a fluid that produces a predetermined response from the analyte sensor, and further comprising determining the response of the analyte sensor to maintenance fluid, and correcting determinations of analyte properties of blood to correct for analyte sensor performance indicated by a comparison of the actual analyte sensor response to the maintenance fluid with the predetermined response of the analyte sensor.

25. A method as in claim 17, wherein the apparatus further comprises a pressure sensor responsive to fluid pressure in the apparatus, and wherein the method further comprises adjusting the fluid pump operation to prevent fluid pressure in the apparatus from exceeding a predetermined pressure.

26. A method as in claim 17, further comprising, at a time when not operating according to steps a) through by, operating the first and second fluid control systems to push a maintenance fluid into the blood removal element at a rate sufficient to encourage the access to the patient's circulatory system to remain open.

27. A method as in claim 17, wherein the apparatus further comprises a pressure sensor operatively connected to at least a portion of the fluid paths between or within the elements of the apparatus, and wherein the operation of the first and second fluid control systems is controlled responsive to the pressure sensor to prevent occlusions from damaging the performance of the system.

28. A method as in claim 17, wherein the apparatus further comprises a pressure sensor operatively connected to at least a portion of the fluid paths between or within the elements of the apparatus, and wherein the presence of air in a portion of the apparatus is determined from the pump operation and the pressure sensor.

29. An apparatus as in claim 6, further comprising an air embolus detector operatively connected with at least one of the fluid paths in the apparatus.

30. An apparatus as in claim 6, further comprising a pressure sensor operatively connected with at least one of the fluid paths in the apparatus.

31. An apparatus as in claim 6, further comprising a blood leak detector operatively connected with at least one of the fluid paths in the apparatus.

32. A blood access system comprising:
a. A junction in fluid communication with a catheter;
b. A first pinch valve in fluid communication with a first side of the junction;
c. A second pinch valve in fluid communication with a second side of the junction;
d. An analyte sensor in fluid communication with the first pinch valve and the second pinch valve;
e. A first pump in fluid communication with the first pinch valve and the analyte sensor;
f. A second pump in fluid communication with the second pinch valve and the analyte sensor;
g. A maintenance fluid container connection in fluid communication with the firs pump; and
h. A waste channel in fluid communication with the second pump.

33. A blood access system comprising:
a. A junction in fluid communication with a catheter;
b. A first pinch valve having a first side in fluid communication with a first side of the junction;
c. A second pinch valve having a first side in fluid communication with a second side of the junction;
d. A first intermediate fluid section in fluid communication with a second side of the first pinch valve;
e. A second intermediate fluid section in fluid communication with a second side of the second pinch valve;
f. An analyte sensor having a first side in fluid communication with the first intermediate fluid section and having a second side in fluid communication with the second intermediate fluid section;
g. A first pump having a first side in fluid communication with the first intermediate fluid section;
h. A second pump having a first side in fluid communication with the second intermediate fluid section;
i. A maintenance fluid container connection in fluid communication with a second side of the first pump; and
j. A waste channel in fluid communication with a second side of the second pump.
